(19) United States
(12) Patent Application Publication
(10) Pub. No.: US 2020/0345707 A1
(43) Pub. Date: Nov. 5, 2020

(54) STRUCTURAL BASIS FOR OUTER MEMBRANE USHER ACTIVATION TO CATALYZE P PILUS ASSEMBLY

(71) Applicant: Washington University, St. Louis, MO (US)

(72) Inventors: Scott HULTGREN, St. Louis, MO (US); Natalie OMATTAGE, St. Louis, MO (US); Fredrik ALMQVIST, St. Louis, MO (US); Jerome PINKNER, St. Louis, MO (US); Karen DODSON, St. Louis, MO (US); Peng YUAN, St. Louis, MO (US)

(21) Appl. No.: 16/773,531

(22) Filed: Jan. 27, 2020

Related U.S. Application Data
(60) Provisional application No. 62/796,966, filed on Jan. 25, 2019.

(51) Int. Cl. A61K 31/4365; A61K 31/5377

(52) U.S. Cl. CPC A61K 31/4365; A61K 45/06; A61K 31/5377

(57) ABSTRACT

Many Gram-negative pathogens assemble adhesive pili structures on their surfaces that allow them to colonize host tissues and cause disease. The present invention relates to novel compounds that disrupt the intramolecular interactions of the pilus subunits thereby reducing pilus assembly.

### CROSS REFERENCE TO RELATED APPLICATIONS

This application claims the benefit of U.S. Provisional Application 62/796,966, filed Jan. 25, 2019, the disclosure of which is hereby incorporated by reference in its entirety.

### GOVERNMENTAL RIGHTS

This invention was made with government support under AI029549 and AI048689 awarded by the National Institutes of Health. The government has certain rights in the invention.

### FIELD OF THE TECHNOLOGY

This disclosure generally relates to compounds and methods for the treatment of diseases caused by tissue-adhering pilus-forming bacteria. More specifically, the invention relates to pharmaceutical preparations comprising substances capable of interfering with the binding of periplasmic chaperones to pilus subunits as well as pharmaceutical compounds capable of interfering with the binding between pilus subunits.

### BACKGROUND

Many pathogenic Gram-negative bacteria such as Escherichia coli, Haemophilus influenzae, Salmonella enteriditis, Salmonella typhimurium, Bordetella pertussis, Yersinia enterocolitica, Yersinia perstis, Helicobacter pylori and Klebsiella pneumoniae assemble hair-like adhesive organelles called pili on their surfaces. Pili are thought to mediate microbial attachment, often the essential first step in the development of disease, by binding to receptors present in host tissues and may also participate in bacterial-bacterial interactions important in biofilm formation.

Uropathogenic strains of E. coli express P and type 1 pili that bind to receptors present in uroepithelial cells. Adhesive P pili are virulence determinants associated with pyelonephritic strains of E. coli whereas type 1 appear to be more common in E. coli causing cystitis. The adhesin present at the tip of the pilus, PapG binds to the Gal (1-4)Gal moiety present in the glycolipids and glycoproteins, while the type 1 adhesin, FimH, binds D-mannose present in glycolipids and glycoproteins.

Type 1 pili are adhesive fibers expressed in E. coli as well as in most of the Enterobacteriaceae family. The type 1 pilus is a right handed helix with about 3 subunits per turn, a diameter of approximately 70 Å, a central pore of about 20-25 Å, and a rise per subunit of about 8 Å. See G. E. Soto et al., EMBO J, 17: 6155 (1998). Type 1 pili are composite structures in which a short tip fibrillar structure containing FimG and the FimH adhesin (and possibly the minor component FimF as well) are joined to a rod comprised predominantly of FimA subunits. See Jones et al., Proc. Natl. Acad. Sci. U.S.A., 92: 2081 (1995). The FimH adhesin mediates binding to mannose-oligosaccharides. See S. N. Abraham et al., Nature, 336: 682 (1988); K. A. Krogfelt et al., Infect. Immun., 58: 1995 (1990). In uropathogenic E. coli, this binding event has been shown to play a critical role in bladder colonization and disease.

Type 1 pilus biogenesis proceeds by way of a highly conserved chaperone/usher pathway that is involved in the assembly of over 25 adhesive organelles in the Gram-negative bacteria. See G. E. Soto and S. Hultgren, J. Bacteriol., 181: 1059 (1999). The usher forms an oligomeric channel in the outer membrane with a pore size of approximately 2.5 nm and mediates subunit translocation across the outer membrane. See D. G. Thanassi et al., Proc. Natl. Acad. U.S.A., 95: 3146 (1998).

P pili is a heteropolymeric surface fiber with an adhesive tip and consists of two major sub-assemblies, the pilus rod and the tip fibrillum. The pilus rod is a thick rigid rod made up of repeating PapA subunits arranged in a right-handed helical cylinder whereas the tip fibrillum is a thin, flexible tip fiber extending from the distal end of the pilus rod and is composed primarily of repeating PapE subunits arranged in an open helical configuration. Two components of the tip fibrillum, PapK and PapF, act as adaptors. PapK is thought to link the pilus rod to the base of the tip fibrillum and regulates the length of the tip fibrillum: its incorporation terminates its growth and nucleates the formation of the pilus rod. PapF is thought to join the PapG adhesin to the distal end of the flexible tip fibrillum.

The biogenesis of P pili also occurs via the highly conserved chaperone/usher pathway. See T. G. Thanassi et al., Curr. Opin. Microbiol., 1: 223 (1998); D. L. Hung et al., EMBO J, 15: 3792 (1996). P pili are adhesive organelles encoded by eleven genes in the pap (pilus associated with pyelonephritis) gene cluster found on the chromosome of uropathogenic strains of E. coli. Six genes encode structural pilus subunits, PapA, PapH, PapK, PapE, PapF and PapG. See S. J. Hultgren et al., Cell 73: 887 (1993).

In P pili, two of the genes in the pap operon, papD and papC, encode the chaperone and usher, respectively. Chaperones such as PapD in E. coli are required to bind to pilus proteins imported into the periplasmic space, partition them into assembly component complexes and prevent non-productive aggregation of the subunits in the periplasm. See Kuehn M. J. et al., Proc. Natl. Acad. Sci. USA 88: 10586 (1991). PapD is a periplasmic chaperone that mediates the assembly of P pili. Detailed structural analysis has revealed that the PapD chaperone is the prototype member of a conserved family of periplasmic chaperones in Gram-negative bacteria. Periplasmic chaperones consist of two immunogloblin-like domains with a deep cleft between the two domains. See A. Holmgren and C. I. Branden, Nature, 342: 248 (1989); M. Pellecchia et al., Nature Struct. Biol., 5: 885 (1998). Further, all members of the periplasmic chaperone superfamily have a conserved hydrophobic core that maintains the overall features of the two domains.

Periplasmic chaperones, along with outer membrane ushers, constitute a molecular mechanism necessary for guiding biogenesis of adhesive organelles in Gram-negative bacteria. These chaperones function to cap and partition interactive subunits imported into the periplasmic space into assembly competent co-complexes, making non-productive interactions unfavorable. The chaperone-subunit co-complexes are targeted to the outer membrane usher where subunits, or ushers, assemble in a specific order to form a pilus. During pilus biogenesis, PapD binds to and caps interactive surfaces on pilus subunits and prevents their premature aggregation in the periplasm. PapD binds to each of the pilus subunit types as they emerge from the cytoplasmic membrane and escorts them in assembly-competent, native-like conformations from the cytoplasmic membrane to outer membrane assembly sites comprised of PapC. PapC has been termed a molecular usher since it receives chaperone-subunit co-complexes and incorporates, or ushers, the subunits from the chaperone co-complex into the growing pilus in a defined order.

In the absence of an interaction with the chaperone, pilus subunits aggregate and are proteolytically degraded. Kolmer et al. and Jones et al. have shown that the DegP protease degrades pilus subunits in the absence of the chaperone. See J. Bacteriol. 178: 5925 (1996); BIBO, 16: 6394 (1997). This discovery led to the elucidation of the fate of pilus subunits expressed in the presence or absence of the chaperone using monospecific antisera in Western blots of cytosolic membrane, outer membrane and perplasmic proteins prepared according to methods known in the art.

Thus, prevention or inhibition of normal pilus assembly in Gram-negative bacterium impacts the pathogenicity of the bacterium by preventing the bacterium from attaching to and infecting host tissues. Moreover, changes in the binding between pilus subunits and chaperones can have a dramatic impact on the efficiency of pilus assembly, and thus on the ability of Gram-negative bacterium to adhere to and consequentially, infect host tissues. Prevention and inhibition of binding between pilus subunits and between pilus subunits and periplasmic chaperones have the effect of impairing pilus assembly, whereby the infectivity of the Gram-negative bacterium expressing the pili is reduced.

Accordingly, a need exists, in general, for compositions and methods for preventing or inhibiting the normal interaction between pilus subunits and/or between a pilus subunit and a chaperone.

### SUMMARY

One aspect of the present disclosure relates to method of treating or preventing a bacterial infection in a subject in need thereof, the method comprising administering to the subject an effective amount of a composition comprising a compound that prevents or inhibits the interaction between an usher protein's amino-terminal periplasmic domain (NTD) and carboxy-terminal periplasmic domain 2 (CTD2). In some embodiments, the compound is a bicyclic 2-pyridone compound comprising formula (I):

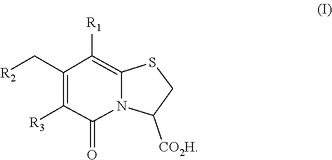

wherein R₁, R₂, and/or R₃ substituents directly disrupt the tripartite interface
comprised of the PapC NTD, PapC CTD2 and PapD.

In some embodiments, the substituents at R2 and/or R3 directly disrupt the NTD-CTD2 interface and mimic sidechains of residues S22 and F21 from the NTD. In another embodiment, the substituent at R₂ introduces steric hindrance at the NTD-CTD2 interface. In still another embodiment, the substituent at R₁ direct disrupts the N-terminal tail a-helix.

One aspect of the present disclosure relates to method of treating or preventing a bacterial infection in a subject in need thereof, the method comprising administering to the subject an effective amount of a composition comprising a comprising formula (Ia):

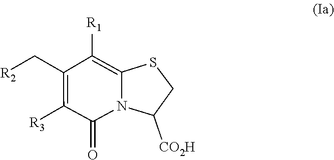

wherein:
R₁ is independently selected from H, C₃₋₆ cycloalkyl, or substituted phenyl group;
R₂ is independently selected from H or aryl; and
R₃ is independently selected from H or —CH₂-X, wherein X is a heterocyclic group.

In some embodiments, R₁ is

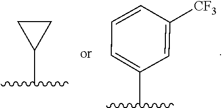

In some embodiments, R₂ is

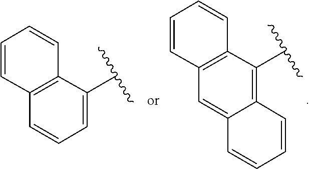 [SMILES: *c1cccc2ccccc12 ; *c1c2ccccc2cc2ccccc12]

In some embodiments, R₃ is H or

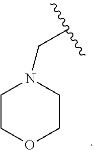 [SMILES: *CN1CCOCC1]

In some embodiments, the compound is

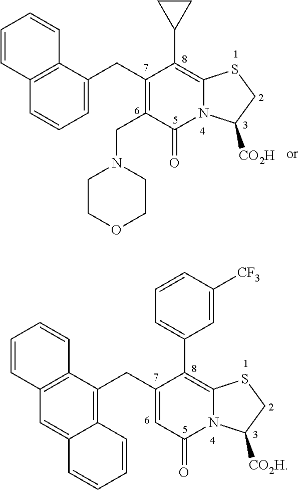 [SMILES: O=C(O)[C@@H]1CSc2c(C3CC3)c(Cc3cccc4ccccc34)c(CN3CCOCC3)c(=O)n21 ; O=C(O)[C@@H]1CSc2c(-c3cccc(C(F)(F)F)c3)c(Cc3c4ccccc4cc4ccccc34)cc(=O)n21]

In some aspects, the compound blocks chaperone-adhesion translocation, thereby inhibiting pilus biogenesis. In some embodiments, the method of treating an infection does not include administration of an antibiotic. In other embodiments, the method of treating includes administration of an antibiotic.

In some embodiments, the disclosure provides method of treating a bacterial infection where the bacteria comprise a chaperone-usher pathway pilus. In some embodiments, the bacteria are gram-negative bacteria.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A-1D show the structure of the PapC-PapDG ternary complex. FIG. 1A provides a schematic diagram of domain organization of PapC, PapD (PapD-D1, N-terminal domain PapD-D2, C-terminal domain), and PapG (PapG_{L}, lectin domain PapG_{P}, pilin domain). FIG. 1B shows a in vitro reconstitution of PapC-PapDG yields stable ternary complex indicated by size-exclusion chromatography. SDS-PAGE shows that Peak A represents the PapC-PapDG ternary complex and Peak B represents excess PapDG. FIG. 1C shows donor-strand exchange (DSE) assay demonstrating that the purified PapC-PapDG complex is functional. PapC-PapDG was challenged at t=0 with PapDF. SDS-PAGE visualized formation of PapG-PapF intermediate at each collected time point (see Methods below for detail). n=3 replicates. FIG. 1D provides the overall architecture of the PapC-PapDG ternary complex. Domains are distinctly colored.

FIG. 2A-2G show a Comparison of the pre-activation PapC-PapDG and the post-activation FimD-FimCH structures. FIG. 2A is a side-view of PapC-PapDG and FimD-FimCH (PDB ID: 3RFZ) structures. The chaperones (PapD and FimC), adhesins (PapG and FimH) and ushers (PapC and FimD) are colored identically. FIG. 2B-FIG. 2E show domain movements required for transition from the pre-activation (PapC-PapDG) usher state to the post-activation (FimD-FimCH) usher state (PDB ID: 3RFZ). FimD-FimCH is depicted in grey. Domain movements between the two conformational states were calculated using the DynDom protein domain analysis server. The reported translation/rotation values for the dynamic domains were the following: (FIG. 2B) PD (20 Å/114°); (FIG. 2C) NTD (13 Å/47°), (FIG. 2D) CTD2 (19 Å/87°) and (FIG. 2E) PapG_{P} (19 Å/87°). FIG. 2F shows a schematic diagram of a proposed model of usher activation for P pilus biogenesis. 1) P Pilus assembly begins with recruitment of chaperone-adhesin (PapDG) to the N-terminal tail of the usher NTD (inactive state). 2) Upon recruitment of chaperone-adhesin to the N-terminal tail of the NTD, CTD2 extends into periplasm to bind to chaperone and the NTD (pre-activation state). Next, 3) Recruitment of the subsequent chaperone-subunit (PapDF) to the N-terminal tail of the NTD might result in concomitant 4) displacement of PD 5) complete translocation of chaperone-adhesin to CTDs and 6) insertion of the adhesin into the TD lumen (activation state). The usher is then activated to serve as an assembly platform to promote DSE reactions between subunits (post-activation state). Alternatively, after pre-activation of the usher (red arrow), 3) translocation of PapDG to the CTDs might elicit 4) PD displacement followed by 5) insertion of the adhesion into the TD lumen (activation state). 6) The NTD-PD complex is then free for recruitment of PapDF to continue the assembly process (post-activation state). FIG. 2G shows shows PapDGp structure remains unchanged in the PapC usher-bound state. Superposition of apo-PapDGp (colored grey; PDB ID: 2WMP) and PapDGp (colored orange and green, respectively) from the PapC-PapDG structure reveals that the overall structure is nearly identical (Ca r.m.s.d.=0.839 A).

FIG. 3A-3E show tripartite interface between PapD and the NTD and CTD2 of PapC. FIG. 3A shows NTD-PapD interface. F3 of the PapC NTD interacts with a hydrophobic pocket comprised of P30, L32, 193, P95 of the PapD chaperone. Chaperone residues are colored green for clarity. FIG. 3B shows CTD2-PapD interface. A salt bridge is formed between K44 of PapD and D762 of PapC CTD2. Side chains are colored by atom types. FIG. 3C shows NTD-CTD2 interface. Residues that play an important role at this interface, as measured by hemagglutination titer analysis (FIG. 3D) are highlighted. FIG. 3D shows hemagglutination assays were performed to test the ability of PapC mutants to complement a ΔpapC pap operon (papAHDJKEFG) for assembly of adhesive P pili on the bacterial surface. The HA titer represents the highest fold dilution of bacteria capable of agglutinating human red blood cells. Titers are represented as the reciprocal of the endpoint dilution. Data are represented as mean±s.e.m. Statistical analyses were performed using unpaired two-tailed Mann-Whitney test; ***P=0.0002 and ***P=0.0009 (T19P PapC). n=8 replicates. Replicates are biological. FIG. 3E shows the proposed mechanism of subunit transfer from NTD to CTD2. Superposition of PapC-PapDG with FimD NTD-FimC-FimH_{P} (PDB ID: 1ZE3) demonstrates that CTD2 would directly clash with the NTD as it interacts with chaperone-adhesin during the initial targeting step. Subunit transfer may occur when CTD2 reaches over to interact with the N-terminal tail and chaperone (1), which results in displacement of the NTD from the main interaction interface (2)

FIG. 4A-4F show PapC NTD and CTD2 mutants are defective for usher activation. FIG. 4A shows the in vitro reconstitution of NTD mutated PapC-PapDG does not yield stable ternary complex indicated by size-exclusion chromatography. SDS-PAGE shows that Peak A represents the mutant PapC alone and Peak B represents excess PapDG. FIG. 4B shows the in vitro reconstitution of NTD mutated PapC-PapDG does not yield stable ternary complex indicated by size-exclusion chromatography. SDS-PAGE shows that Peak A represents the mutant PapC alone and Peak B represents excess PapDG. FIG. 4C shows the in vitro reconstitution of NTD mutated PapC-PapDG does not yield stable ternary complex indicated by size-exclusion chromatography. SDS-PAGE shows that Peak A represents the mutant PapC alone and Peak B represents excess PapDG n=1 replicate. FIG. 4D shows the in vitro reconstitution of CTD2 mutated PapC-PapDG yields stable ternary complex indicated by size-exclusion chromatography. SDS-PAGE shows that Peak A represents the mutant PapC-PapDG ternary complex and Peak B represents excess PapDG. n=1 replicate. FIG. 4E shows the donor-strand exchange (DSE) assay demonstrates F745A PapC-PapDG is less efficient at promoting DSE at each time point compared to wild-type PapC-PapDG (FIG. 1C). F745A PapC-PapDG was challenged at t=0 with PapDF. SDS-PAGE analysis visualized formation of PapG-PapF intermediate at each collected time point. FIG. 4F shows the quantification of PapG-PapF band at t=48 h demonstrates reduction in F745A PapC-PapDG mediated DSE relative to wild-type PapC-PapDG.

FIG. 5A-5D show electron density in the NTD N-terminal tail region (1-24) and that the CTD2-chaperone interface is conserved between type 1 and P pilus systems. FIG. 5 A shows the weighted 2Fo-Fc electron density at 3.7 A following refinement was contoured at 1.0 a. Residues with notable sidechain density are labeled for clarity. FIG. 5B shows a superposition of PapC CTD2-PapD chaperone (colored purple and orange, respectively) and FimD CTD2-FimC chaperone (colored grey; PDB ID: 3RFZ) reveals similar interface. FIG. 5C shows PapC CTD2-PapD chaperone. FIG. 5D shows FimD CTD2-FimC chaperone shown at a similar orientation. CTD2 13-strands are denoted.

FIG. 6 shows HA titer analysis of additional PapC mutants. Mutation of residues outside of the NTD-CTD2 interface does not affect P pilus assembly. Hemagglutination assays were performed to test the ability of PapC mutants to complement a ApapC pap operon (papAHDJKEFG) for assembly of adhesive P pili on the bacterial surface. The HA titer represents the highest fold dilution of bacteria capable of agglutinating human red blood cells. Titers are represented as the reciprocal of the endpoint dilution. Data are represented as mean±s.e.m. n=3 replicates. Replicates are biological.

FIG. 7A-7B show the proposed mechanism of action for small molecule inhibitors of pilus assembly. FIG. 7A shows superposition of PapC-PapDG with PapD: NP048 structure (PDB ID: 2J7L) reveals that pilicide NP048 directly disrupts the tripartite interface comprised of the PapC NTD, PapC CTD2 and PapD. The morpholine and CH₂-1-naphthyl substituents at C-6 and C-7 of the pilicide, respectively, directly clash with this NTD-CTD2 interface and specifically mimic sidechains of residues S22 and F21 from the NTD. FIG. 7B shows superposition of ec312 with NP048 reveals that the C-7 anthracene substituent would introduce an even greater steric hindrance at the NTD-CTD2 interface than NP048, and that the C-8 CF3 substituent would likely clash with the N-terminal tail a-helix.

### DETAILED DESCRIPTION

Identification of such compositions which have the ability to disrupt pillus assembly has been elusive. Previously identified compounds were ether discovered by serendipity and/or systematic screening of large numbers of natural and synthetic compounds. A far superior method of drug-screening relies on structure-based drug design. The three dimensional structures of proteins or protein fragments are determined and potential agonists and/or potential antagonists are designed with the aid of computer modeling. However, prior to the present disclosure three-dimensional structure illustrating the interaction between pilus subunits and/or between domains of the same pilus subunit has remained unknown, essentially because no such protein co-crystals had been produced which would permit the required X-ray crystallographic data to be obtained.

The present disclosure is based, at least in part, on the discovery of the mechanism by which the outer membrane usher transfers substrates from its N-terminus to C-terminus via an unexpected interaction between two periplasmic domains. In particular, the present disclosure provides insight into the NTD-CTD2 interaction of PapC and establishes the intramolecular interaction to be essential for stable association of PapDG with the PapC usher and translocation of subunits from the NTD to CTDs. As described herein, CTD2 drives subunit transfer, potentially by using the avidity of its interactions with both the NTD and PapD to competitively displace subunits from the NTD during pilus biogenesis. Compositions which sterically block the NTD-CTD2 interface can selectively hinder the usher from acting as an assembly platform. Therefore, the present disclosure provides, inter alia, methods and compositions for inhibiting or reducing pilus biogenesis by blocking chaperone-adhesion translocation. In addition, the present disclosure provides method and compositions which are useful for treating or preventing a bacterial infection in a subject. Moreover, the present disclosure provides an in vitro method of screening candidate molecules capable of reducing pilus assembly.

Additional aspects of the disclosure are described below.

### (I) Compositions

The present disclosure provides antibacterial compositions and compounds capable of inhibiting or preventing pilus assembly in a Gram-negative bacterium. Such compounds interfere with the function of chaperones required for the assembly of pili from pilus subunits in diverse Gram-negative bacteria. Another object of the disclosure is to provide compounds having antibacterial activity that prevent or inhibit pili assembly by interfering with the interactions between pilus subunits and/or between domains of the same pilus subunit (e.g. intramolecular interactions). Yet another object of the invention is to provide compounds capable of inhibiting or preventing the function of pili adhesion to host epithelium thereby reducing the capacity of bacteria to attach to and infect host tissues. It is a further object of the invention to provide antibacterial compounds which have broad specificity for a diverse group of Gram-negative bacteria. Other objects include the provision of methods of preventing and inhibiting pilus assembly, methods of preventing or inhibiting pili adhesion to host tissues, methods of treating bacterial infections, methods for preventing and inhibiting biofilm formation and methods of preventing colonization by various Gram-negative bacterium.

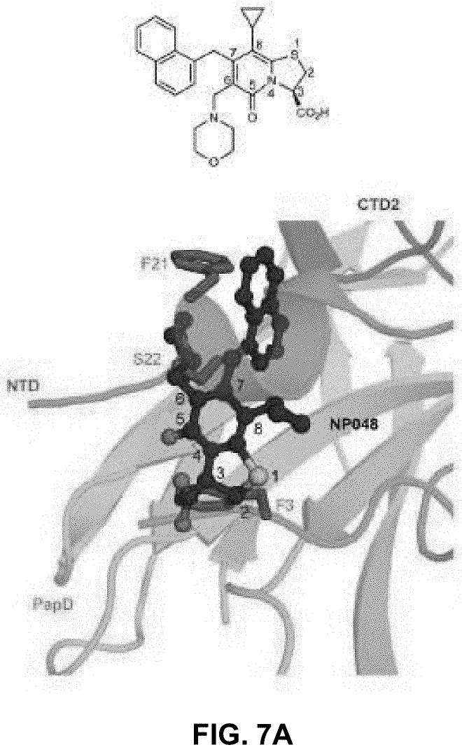

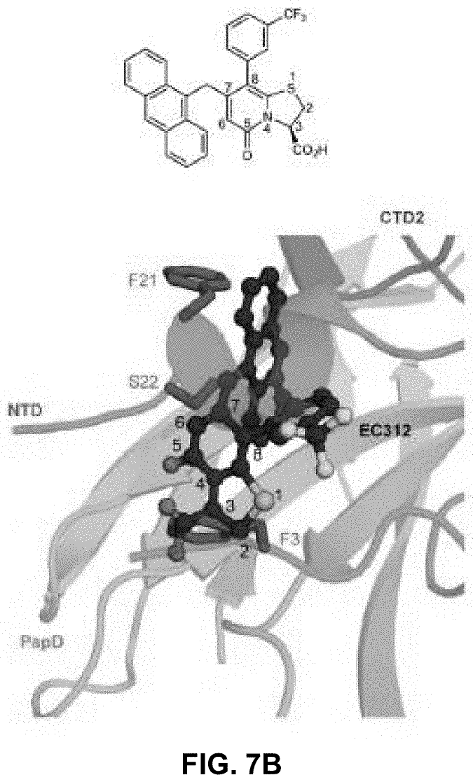

One aspect of the present disclosure is directed to the provision of a composition comprising a compound which blocks chaperone-adhesion translocation, thereby inhibiting pilus biogenesis. In some embodiments, the compound prevents or inhibits the interaction between an usher protein's amino-terminal periplasmic domain (NTD) and carboxy-terminal periplasmic domain 2 (CTD2). For example, the compound can prevent or inhibit the interaction between the subunit domains by sterically blocking the NTD-CTD2 pilus domain interface to selectively hinder the usher from acting as an assembly platform. In some embodiments, the compound prevents or inhibits the N-terminal tail residues 12-21 of the NTD from forming interactions with the bottom cleft of CTD2, particularly the β1-β2 and β5-β6 loops in CTD2 (see, e.g. FIG. 7). In one aspect, the compound prevents or inhibits the interface between residues 12-22 of NTD and CTD2. In another aspect, the compound prevents or inhibits the interface between residues 12-21 of NTD and CTD2. In still another aspect, the compound prevents or inhibits the interface between residues 20-22 of NTD and CTD2. In some embodiments, the compound mimics the side chains of residues S22 and F21 of NTD thereby preventing or inhibiting the NTD-CTD2 interface.

As utilized herein, the term “pilus” or “pili” relates to fibrillar heteropolymeric structures embedded in the cell envelope of many tissue-adhering pathogenic bacteria, notably pathogenic gram negative bacteria. In the present specification, the terms pilus and pili will be used interchangeably. A pilus is composed of a number of “pilus subunits” which constitute distinct functional parts of the intact pilus.

The term “chaperone” relates to a molecule which in living cells has the responsibility of binding to polypeptides in order to mature the polypeptides in a number of ways. Many molecular chaperones are involved in the process of folding polypeptides into their native conformations whereas other molecular chaperones are involved in the export out of or import into the cell of polypeptides. Specialized molecular chaperones are “periplasmic chaperones” which are bacterial molecular chaperones exerting their main actions in the “periplasmic space.” Specialized periplasmic chaperones also have an immunoglobulin-like three dimensional structure. The periplasmic space constitutes the space in between the inner and outer bacterial membrane. Periplasmic chaperones are involved in the process of correct assembly of intact pili structures. When used herein, the use of the term “chaperone” designates a molecular, periplasmic chaperone unless otherwise indicated.

The phrase “preventing or inhibiting binding between pilus subunits” generally indicates that the normal interaction between pilus subunits is being affected either by being inhibited, disrupted protein-protein interactions (e.g., electrostatic forces, hydrogen bonding and the hydrophobic effect), or reduced to such an extent that the binding of a pilus subunit to another pilus subunit is measurably lower than is the case when the pilus subunits are interacting at conditions which are substantially identical (with regard to pH, concentration of ions, and other molecules) to the native conditions during pilus assembly. This phrase can apply to the dissociation of pre-formed pilus subunit—subunit interactions during pilus assembly. Measurement of the degree of binding can be determined in vitro by methods known to the person skilled in the art (microcalorimetry, radioimmunoassays, enzyme based immunoassays, etc.).

The phrase “preventing or inhibiting binding between pilus subunit domains” generally indicates that the normal interaction between pilus subunits domains is being affected either by being inhibited, disruption of protein-protein interactions (e.g., electrostatic forces, hydrogen bonding and the hydrophobic effect), or reduced to such an extent that the binding of a pilus subunit domain to another domain of the same or different pilus subunit (e.g., NTD and CTD2 of PapC) is measurably lower than is the case when the pilus subunits domains are interacting at conditions which are substantially identical (with regard to pH, concentration of ions, and other molecules) to the native conditions during pilus assembly. This phrase can apply to the dissociation of pre-formed pilus subunit domain—subunit domain interactions during pilus assembly. Measurement of the degree of binding can be determined in vitro by methods known to the person skilled in the art (for example, the assays as described in the Examples).

In one aspect, the compound of the disclosure is a bicyclic 2-pyridone compound comprising formula (I):

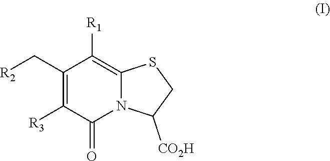

In one embodiment, R₁, R₂, and/or R₃ are a substituent which disrupts pilus biogenesis. For example, R₁, R₂, and/or R₃ can be any substituent which prevents or inhibits pilus subunit interaction. In some embodiments, R₁, R₂, and/or R₃ can be any substituent which prevents or inhibits pilus subunit interaction between an usher protein's NTD and CTD2 domains. In a specific embodiment, the R₁, R₂, and/or R₃ substituents of formula I directly disrupts the tripartite interface comprised of the PapC NTD, PapC CTD2 and PapD. In one embodiment, a substituent at R₂ and/or R₃ of formula I (e.g., a CH₂-1-naphthyl) will directly clash with this NTD-CTD2 interface and specifically mimic sidechains of residues S22 and F21 from the NTD. In another embodiment, a substituent at R₂ of formula I (e.g., an anthracene) introduces steric hindrance at the NTD-CTD2 interface. In still another embodiment, a substituent at R₁ (e.g. phenyl-CF₃) clashes with the N-terminal tail a-helix. Disruption of pilus biogenesis can be determined with HA titer assays as described in Slonim L N et al., EMBO J 11, 4747-4756 (1992).

In one aspect, the compound of the disclosure is a bicyclic 2-pyridone compound comprising formula (Ia):

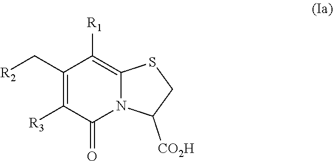

wherein:
R₁ is independently selected from H, C₃₋₆ cycloalkyl, or substituted phenyl group;
R₂ is independently selected from H or aryl; and
R₃ is independently selected from H or —CH₂—X, wherein X is a heterocyclic group.

In an embodiment, a compound of Formula (I) comprises any of the preceding compounds of Formula (I), wherein R₁ is

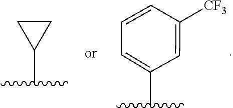

In another embodiment, a compound of Formula (I) comprises any of the preceding compounds of Formula (I), wherein R₂ is

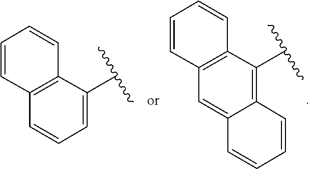 [SMILES: *c1cccc2ccccc12 ; *c1c2ccccc2cc2ccccc12]

In another embodiment, a compound of Formula (I) comprises any of the preceding compounds of Formula (I), wherein R₃ is H or

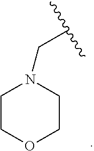 [SMILES: *CN1CCOCC1]

In exemplary embodiments, a compound of the disclosure comprises Formula (I) as shown below:

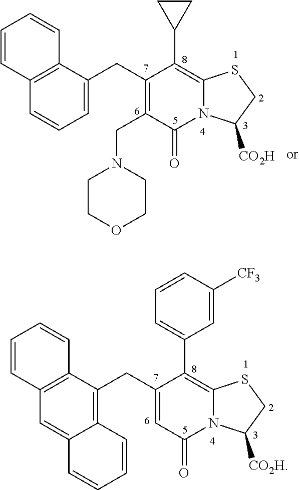 [SMILES: O=C(O)[C@@H]1CSc2c(C3CC3)c(Cc3cccc4ccccc34)c(CN3CCOCC3)c(=O)n21 ; O=C(O)[C@@H]1CSc2c(-c3cccc(C(F)(F)F)c3)c(Cc3c4ccccc4cc4ccccc34)cc(=O)n21]

Pharmaceutical acceptable salts of a compound of the disclosure include, without limit, acetate, aspartate, benzoate, bitartrate, citrate, formate, gluconate, glucuronate, glutamate, fumarate, hydrochloride, hydrobromide, hydroiodide, hypophosphite, isobutyrate, isocitrate, lactate, malate, maleate, meconate, methylbromide, methanesulfonate, monohydrate, mucate, nitrate, oxalate, phenylpropionate, phosphate, phthalate, propionate, pyruvate, salicylate, stearate, succinate, sulfate, tannate, tartrate, terephthalate, valerate, and the like.

### (a) Components of the Composition

The present disclosure also provides pharmaceutical compositions. The pharmaceutical composition comprises a compound as described above, as an active ingredient, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

In each of the embodiments described herein, a composition of the invention may optionally comprise one or more additional drug or therapeutically active agent in addition to the compound of the disclosure. Thus, in addition to the therapies described herein, one may also provide to the subject other therapies known to be efficacious for treatment of the bacterial infection. In some embodiments, the additional drug or therapeutically active agent is an antibiotic such as erythromycin, tetracycline, macrolides, for example azithromycin and the cephalosporins. Depending on the mode of administration, the compounds will be formulated into suitable compositions to permit facile delivery to the affected areas.

(i) Diluent

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

(ii) Binder

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

(iii) Filler

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

(iv) Buffering Agent

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

(v) pH Modifier

In various embodiments, the excipient may be a pH modifier. By way of non-limiting example, the pH modifying agent may be sodium carbonate, sodium bicarbonate, sodium citrate, citric acid, or phosphoric acid.

(vi) Disintegrant

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

(vii) Dispersant

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

(viii) Excipient

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

(ix) Lubricant

In a further embodiment, the excipient may be a lubricant. Non-limiting examples of suitable lubricants include minerals such as talc or silica; and fats such as vegetable stearin, magnesium stearate, or stearic acid.

(x) Taste-Masking Agent

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

(xi) Flavoring Agent

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

(xii) Coloring Agent

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

### (b) Administration

(i) Dosage Forms

The composition can be formulated into various dosage forms and administered by a number of different means that will deliver a therapeutically effective amount of the active ingredient. Such compositions can be administered orally (e.g. inhalation), parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, intra-articular, or intrasternal injection, or infusion techniques. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980). In a specific embodiment, a composition may be a food supplement or a composition may be a cosmetic.

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof. For administration by inhalation, the compounds are delivered in the form of an aerosol spray from pressured container or dispenser which contains a suitable propellant, e.g., a gas such as carbon dioxide, or a nebulizer.

For parenteral administration (including subcutaneous, intradermal, intravenous, intramuscular, intra-articular and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules, and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols, or oils. In some embodiments, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles, and mouth washes. Transmucosal administration may be accomplished through the use of nasal sprays, aerosol sprays, tablets, or suppositories, and transdermal administration may be via ointments, salves, gels, patches, or creams as generally known in the art.

In certain embodiments, a composition comprising the compound of the disclosure, is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present invention. Non-limiting examples of suitable structured fluid delivery systems may include nanoparticles, liposomes, microemulsions, micelles, dendrimers, and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery of the compound of the disclosure, in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the the compound of the disclosure may be selectively delivered to a cell by encapsulation in a liposome that fuses with the targeted cell's membrane.

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tetradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9,12,15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying the compound of the disclosure may be prepared by any known method of preparing liposomes for drug delivery, such as, for example, detailed in U.S. Pat. Nos. 4,241,046; 4,394,448; 4,529,561; 4,755,388; 4,828,837; 4,925,661; 4,954,345; 4,957,735; 5,043,164; 5,064,655; 5,077,211; and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of the compound of the disclosure, concentration and composition of lipid, concentration of multivalent cations, rate of mixing, presence of and concentration of solvent.

In another embodiment, a composition of the invention may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. The compound of the disclosure may be encapsulated in a microemulsion by any method generally known in the art.

In yet another embodiment, the compound of the disclosure, may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

Generally, a safe and effective amount of the compound of the disclosure is, for example, that amount that would cause the desired therapeutic effect in a subject while minimizing undesired side effects. In various embodiments, an effective amount of the compound of the disclosure described herein can substantially blocks chaperone-adhesion translocation, thereby inhibiting pilus biogenesis and treating the bacterial infection.

When used in the treatments described herein, a therapeutically effective amount of the compound of the disclosure can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat or prevent a bacterial infection.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of the compound of the disclosure can occur as a single event or over a time course of treatment. For example, the compound of the disclosure can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a bacterial infection.

The compound of the disclosure can be administered simultaneously or sequentially with another agent, such as an antibiotic, an anti-inflammatory, or another agent. For example, the compound of the disclosure can be administered simultaneously with another agent, such as an antibiotic or an anti-inflammatory. Simultaneous administration can occur through administration of separate compositions, each containing one or more of the compound of the disclosure, an antibiotic, an anti-inflammatory, or another agent. Simultaneous administration can occur through administration of one composition containing two or more of the compound of the disclosure, an antibiotic, an anti-inflammatory, or another therapeutic agent for an treating a bacterial infection. The compound of the disclosure can be administered sequentially with an antibiotic, an anti-inflammatory, or another agent. For example, the compound of the disclosure can be administered before or after administration of an antibiotic, an anti-inflammatory, or another agent.

### (II) Methods

The present disclosure encompasses a method of treating a bacterial infection in a subject in need thereof. Generally, the method comprises administration of a therapeutically effective amount of a compound of the disclosure, so as to blocks chaperone-adhesion translocation, thereby inhibiting pilus biogenesis. In another embodiment of the disclosure, a method of preventing or inhibiting pilus assembly in bacteria is provided by administering a compound of the disclosure which will prevent or inhibit pilus subunit domain interactions. Also provided is a method of preventing or inhibiting the adhesion of bacteria to a host tissue by administering a compound of the disclosure which will inhibit pilus biogenesis thereby reducing pilus mediated adhesion. Thus, the methods of the present disclosure may be utilized to inhibit pili assembly and/or pili adhesion by providing an effective amount of such compositions to a subject. In still yet another aspect, the present disclosure provides a composition comprising a compound of the disclosure, for use in vitro, in vivo, or ex vivo. Suitable compositions comprising a compound as disclosed herein are, for instance, those described in Section I.

According to an aspect of the invention a pharmaceutical composition comprising a compound of the disclosure can treat, reduce, or prevent a a bacterial infection in a subject. For example, the methods of the disclosure are useful for treating or preventing an infection mediated by a gram-negative bacteria. In another embodiment, the methods of the disclosure are useful for treating or preventing an infection by bacteria comprising a chaperone-usher pathway pilus.

The methods of the present invention also have a variety of industrial uses, well known to those skilled in such arts, relating to their antibacterial properties. In general, these uses are carried out by bringing a biocidal or bacterial inhibitory amount of the compositions of the present disclosure into contact with a surface, environment or biozone containing Gram-negative bacteria so that the composition is able to interact with and thereby interfere with the biological function of such bacteria. For example, such a composition can be used to prevent or inhibit biofilm formation caused by Gram-negative bacteria and to inhibit bacterial colonization by a Gram-negative organism. Compositions may be formulated as sprays, solutions, pellets, powders and in other forms of administration well known to those skilled in such arts.

It will be understood that the above-described methods comprising administration of substances in treating and/or preventing diseases are dependent on the identification or de novo design of substances which are capable of exerting effects which will lead to prevention or inhibition of the interaction between pilus subunits and periplasmic molecular chaperones. It is further important that these substances will have a high chance of being therapeutically active.

Thus clinical experimental trials and animal studies can be undertaken to demonstrate the therapeutic efficacy of peptide mimics and analogues for preventing or inhibiting pilus assembly. The efficacy of such compounds can be shown using methods known in the art, including pilus inhibition and binding assays, specifically ELISA or hemagglutination.

Moreover, the present disclosure provides an in vitro method for screening for a candidate agent substance, the method comprising identifying candidates that prevent or inhibit the interaction between an usher protein's amino-terminal periplasmic domain (NTD) and carboxy-terminal periplasmic domain 2 (CTD2). The method generally comprises, (a) providing a candidate in a biofilm inhibition assay and selecting a candidate agent that has a target EC₅₀ value; (b) optionally (i) identifying the candidate agent from step (a) that reduce PapDG's association with PapC, or (ii) identifying candidate agent from step (a) that reduce pili assembly as assessed by HA titer analysis; and (c) identifying the candidate agent from either step (a) or step (b) that disrupt the interaction between an usher protein's amino-terminal periplasmic domain (NTD) and carboxy-terminal periplasmic domain 2 (CTD2).

In some embodiments, the target EC₅₀ value is an EC₅₀ value of about 500 μM or less, an EC₅₀ of about 250 μM or less, an EC₅₀ value of about 125 μM or less, an EC₅₀ value of about 75 μM or less, an EC₅₀ value of about 50 μM or less, or an EC₅₀ value of about 25 μM or less.

The candidate agents can be selected from the group consisting of proteins, peptides, nucleic acids (e.g., but not limited to, siRNA, anti-miRs, antisense oligonucleotides, and ribozymes), small molecules, nutrients (lipid precursors), and a combination of two or more thereof. In preferred embodiments, the candidate agent prevents or inhibits the interaction between the subunit domains by sterically blocking the NTD-CTD2 pilus domain interface to selectively hinder the usher from acting as an assembly platform. In some embodiments, the candidate agent prevents or inhibits the N-terminal tail residues 12-21 of the NTD from forming interactions with the bottom cleft of CTD2, particularly the β1-β2 and β5-β6 loops in CTD2 (see, e.g. FIG. 7). In one aspect, the candidate agent prevents or inhibits the interface between residues 12-22 of NTD and CTD2. In another aspect, the candidate agent prevents or inhibits the interface between residues 12-21 of NTD and CTD2. In still another aspect, the candidate agent or inhibits the interface between residues 20-22 of NTD and CTD2. In some embodiments, the candidate agent mimics the side chains of residues S22 and F21 of NTD thereby preventing or inhibiting the NTD-CTD2 interface.

The step of identifying whether the test the candidate agent reduces PapDG's association with PapC and/or disrupt the interaction between an usher protein's amino-terminal periplasmic domain (NTD) and carboxy-terminal periplasmic domain 2 (CTD2) can be performed using assays known to the skilled artisan (e.g., those as described in the below Examples).

The compounds and compositions of the present invention which prevent or inhibit binding between pilus subunits or within domains of the same subunit are said to exhibit “antibacterial activity.”

By the term “subject in need thereof” is in the present context meant a subject, which can be any plant or animal, including a human being, who is infected with, or is likely to be infected with, tissue-adhering pilus-forming bacteria which are believed to be pathogenic.

By the term “an effective amount” is meant an amount of the substance in question which will in a majority of patients have either the effect that the disease caused by the pathogenic bacteria is cured or ameliorated or, if the substance has been given prophylactically, the effect that the disease is prevented from manifesting itself. The term “an effective amount” also implies that the substance is given in an amount which only causes mild or no adverse effects in the subject to whom it has been administered, or that the adverse effects may be tolerated from a medical and pharmaceutical point of view in the light of the severity of the disease for which the substance has been given.

As used herein, “treatment” includes both prophylaxis and therapy. Thus, in treating a subject, the compounds of the invention may be administered to a subject already harboring a bacterial infection or in order to prevent such infection from occurring.

By the term “a mimic of a pilus subunit” is meant a compound which has been established to bind to a pilus subunit in a manner which is comparable to the way the pilus the pilus subunits bind to each other or themselves (intramolecular interactions), respectively.

In the present context the terms “an analogue of a pilus subunit” and “a mimic of a pilus subunit” should be understood, in a broad sense, to mean any substance which mimics (with respect to binding characteristics) an effective part of a pilus subunit (e.g. the amino-terminal portion of the pilus subunit). Thus, the analogue or mimic may simply be any other compound regarded as capable of mimicking the binding between pilus subunits or intramolecular binding within the same subunit (e.g., PapC NTD-CTD2 interactions) in vivo or in vitro. In the present context, the pilus subunit, mimic or analogue thereof exhibits at least one binding characteristic relevant for the assembly of pili (e.g., mimics the N-terminal tail residues 12-21 of the NTD interactions with the bottom cleft of CTD2, particularly the β1-β2 and β5-β6 loops in CTD2).

The terms “an analogue of the side chains of residues S22 and F21 of NTD” or “a mimic of the side chains of residues S22 and F21 of NTD” denotes any substance which mimics or has the ability to interact with the CTD2 pilus subunit in a manner which corresponds to the interactions at the NTD-CTD2 interface (e.g, mimics the N-terminal tail residues 22 and/or 21 of the NTD interactions with the bottom cleft of CTD2, for example, the β1-β2 and β5-β6 loops in CTD2).

The term “donor stand complementation” refers to the mechanism by which a chaperone donates its G 1 beta-strand to complete the fold of a pilus subunit.

The term “donor strand exchange” refers to the mechanism by which the amino-terminal extension of a pilus subunit displaces the G 1 beta-strand of a pilus chaperone and subsequently occupies the subunit groove previously occupied by the G 1 beta-strand.

The phrase “having substantially the same three-dimensional structure” refers to a polypeptide that is characterized by a set of atomic structure coordinates that have a root mean square deviation (r.m.s.d.) of less than or equal to about 2 Å when superimposed onto the atomic structure of polypeptide of interest when at least about 50% to 100% of the C a atoms of the coordinates are included in the superposition.

The amino acid notations used herein for the twenty genetically encoded L-amino acids are conventional and are abbreviated as follows:

| Amino Acid | One-Letter Symbol | Three-Letter Symbol |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Gln |
| Glycine | G | Gly |
| Histidine | H | His |
| Isoleucine | I | Ile |
| Leucine | L | Len |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

As used herein, unless specifically delineated otherwise, the three-letter and one-letter amino acid abbreviations designate amino acids in either the D-configuration or the L-configuration. For example, Arg designates D-arginine and L-arginine, and R designates D-arginine and L-arginine.

Unless noted otherwise, when polypeptide sequences are presented as a series of one-letter and/or three-letter abbreviations, the sequences are presented in the N→C direction, in accordance with common practice. As used herein, “C” refers to the alpha carbon of an amino acid residue.

For purposes of determining conservative amino acid substitutions in the various polypeptides described herein and for describing the various peptide and peptide analog compounds, the amino acids can be conveniently classified into two main categories—hydrophilic and hydrophobic—depending primarily on the physical-chemical characteristics of the amino acid side chain. These two main categories can be further classified into subcategories that more distinctly define the characteristics of the amino acid side chains. For example, the class of hydrophilic amino acids can be further subdivided into acidic, basic and polar amino acids. The class of hydrophobic amino acids can be further subdivided into apolar and aromatic amino acids. The definitions of the various categories of amino acids are as follows:

“Hydrophilic amino acid” refers to an amino acid exhibiting a hydrophobicity of less than zero according to the normalized consensus hydrophobicity scale of Eisenberg et al., 1984, J. Mol. Biol. 179:125-142. Genetically encoded hydrophilic amino acids include Thr (T), Ser (S), His (H), Glu (E), Asn (N), Gin (0), Asp (D), Lys (K) and Arg (R).

“Acidic amino acid” refers to a hydrophilic amino acid having a side chain pK value of less than 7. Acidic amino acids typically have negatively charged side chains at physiological pH due to loss of a hydrogen ion. Genetically encoded acidic amino acids include Glu (E) and Asp (D).

“Basic amino acid” refers to a hydrophilic amino acid having a side chain pK value of greater than 7. Basic amino acids typically have positively charged side chains at physiological pH due to association with hydronium ion. Genetically encoded basic amino acids include His (H), Arg (R) and Lys (K).

“Polar amino acid” refers to a hydrophilic amino acid having a side chain that is uncharged at physiological pH, but which has at least one bond in which the pair of electrons shared in common by two atoms is held more closely by one of the atoms. Genetically encoded polar amino acids include Asn (N), Gin (0) Ser (S) and Thr (T).

“Hydrophobic amino acid” refers to an amino acid exhibiting a hydrophobicity of greater than zero according to the normalized consensus hydrophobicity scale of Eisenberg, 1984, J. Mol. Biol. 179:125-142. Genetically encoded hydrophobic amino acids include Pro (P), Ile (I), Phe (F), Val (V), Leu (L), Trp (W), Met (M), Ala (A), Gly (G) and Tyr (Y).

“Aromatic amino acid” refers to a hydrophobic amino acid with a side chain having at least one aromatic or heteroaromatic ring. The aromatic or heteroaromatic ring may contain one or more substituents such as —OH, —SH, —CN, —F, —Cl, —Br, —I, —NO₂, —NO, —NH₂, —NHR, —NRR, —C(O)R, —C(O)OH, —C(O)OR, —C(O)NH₂, —C(O)NHR, —C(O)NRR and the like where each R is independently (C₁-C₆) alkyl, substituted (C₁-C₆) alkyl, (C₂-C₆) alkenyl, substituted (C₂-C₆) alkenyl, (C₂-C₆) alkynyl, substituted (C₂-C₆) alkynyl, (C₅-C₂₀) aryl, substituted (C₅-C₂₀) aryl, (C₆-C₂₆) arylalkyl, substituted (C₆-C₂₆) arylalkyl, 5-20 membered heteroaryl, substituted 5-20 membered heteroaryl, 6-26 membered heteroarylalkyl or substituted 6-26 membered heteroarylalkyl. Genetically encoded aromatic amino acids include His (H), Phe (F), Tyr (Y) and Trp (W).

“Polar amino acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded apolar amino acids include Leu (L), Val (V), Ile (I), Met (M), Gly (G) and Ala (A).

“Aliphatic amino acid” refers to a hydrophobic amino acid having an aliphatic hydrocarbon side chain. Genetically encoded aliphatic amino acids include Ala (A), Val (V), Leu (L) and Ile (I).

“Hydroxyl-substituted aliphatic amino acid” refers to a hydrophilic polar amino acid having a hydroxyl-substituted side chain. Genetically-encoded hydroxyl-substituted aliphatic amino acids include Ser (S) and Thr (T).

The amino acid residue Cys (C) is unusual in that it can form disulfide bridges with other Cys (C) residues or other sulfanyl-containing amino acids. The ability of Cys (C) residues (and other amino acids with —SH containing side chains) to exist in a peptide in either the reduced free —SH or oxidized disulfide-bridged form affects whether Cys (C) residues contribute net hydrophobic or hydrophilic character to a peptide. While Cys (C) exhibits a hydrophobicity of 0.29 according to the normalized consensus scale of Eisenberg (Eisenberg, 1984, supra), it is to be understood that for purposes of the present invention Cys (C) is categorized as a polar hydrophilic amino acid, notwithstanding the general classifications defined above.

As will be appreciated by those of skill in the art, the above-defined categories are not mutually exclusive. Thus, amino acids having side chains exhibiting two or more physical-chemical properties can be included in multiple categories. For example, amino acid side chains having aromatic moieties that are further substituted with polar substituents, such as Tyr (Y), may exhibit both aromatic hydrophobic properties and polar or hydrophilic properties, and can therefore be included in both the aromatic and polar categories. As another example, His (H) has a side chain that falls within the aromatic and basic categories. The appropriate categorization of any amino acid will be apparent to those of skill in the art, especially in light of the detailed disclosure provided herein.

While the above-defined categories have been exemplified in terms of the genetically encoded amino acids, the amino acid substitutions need not be, and in certain embodiments preferably are not, restricted to the genetically encoded amino acids. Indeed, since many of the compounds described herein may be produced synthetically, they may comprise one or more genetically non-encoded amino acids. Thus, in addition to the naturally occurring genetically encoded amino acids, amino acid residues in the core peptide may be substituted with naturally occurring non-encoded amino acids and synthetic amino acids.

Certain commonly encountered amino acids of which the compounds of the invention may be comprised include, but are not limited to, β-alanine (β-Ala) and other omega-amino acids such as 3-aminopropionic acid, 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (Melle); phenylglycine (Phg); cyclohexylalanine (Cha); norleucine (Nle); naphthylalanine (Nal); 4-chlorophenylalanine (Phe(4-C1)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,4-diaminobutyric acid (Dbu); 2,3-diaminobutyric acid (Dab); p-aminophenylalanine (Phe(pNH 2)); N-methyl valine (MeVal); homocysteine (hCys), homophenylalanine (hPhe) and homoserine (hSer); hydroxyproline (Hyp), homoproline (hPro), N-methylated amino acids and peptoids (N-substituted glycines).

The classifications of the genetically encoded and common non-encoded amino acids according to the categories defined above are summarized in Table 1, below. It is to be understood that Table 1 is for illustrative purposes only and does not purport to be an exhaustive list of amino acid residues that can be used in the invention. Additional amino acids may be found in Fasman, 1989, Practical Handbook of Biochemistry and Molecular Biology, CRC Press, Inc., pp. 3-70, and the references cited therein.

TABLE 1
CLASSIFICATIONS OF COMMONLY
ENCOUNTERED AMINO ACIDS

| | Genetically | Non-Genetically |
|---|---|---|
| Classification | Encoded | Encoded |
| Hydrophobic | | |
| Aromatic | H, F, Y, W | Phg, Nal, Thi, Tic, |
| | | Phe(4-C1), Phe(2-F), |
| | | Phe(3-F), Phe(4-F), |
| | | hPhe |
| Apolar | L, V, I, M, G, A, P | t-BuA, t-BuG, MeIle, |
| | | Nle, MeVal, Cha, |
| | | McGly, Aib |
| Aliphatic | A, V, L, I | b-Ala, Dpr, Aib, Aha, |
| | | MeGly, t-BuA, t-BuG, |
| | | MeIle, Cha, Nle, MeVal |
| Hydrophilic | | |
| Acidic | D, E | Dpr, Orn, hArg, |
| Basic | H, K, R | Phe(p-NH₂), Dbu, Dab |
| Polar | C, Q, N, S, T | Cit, AcLys, MSO, |
| | | bAla, hSer |

The term “crystal” refers to a composition comprising a polypeptide in crystalline form. The term “crystal” includes native crystals, heavy-atom derivative crystals and co-crystals, as defined herein.

The term “native crystal” refers to a crystal wherein the polypeptide is substantially pure. As used herein, native crystals do not include crystals of polypeptides comprising amino acids that are modified with heavy atoms, such as crystals of selenomethionine mutants, selenocysteine mutants, etc.

The term “co-complex” refers to a polypeptide in association with one or more additional polypeptides or other molecules. For example, the PapD-PapK and FimC-FimH assemblies are co-complexes.

The term “co-crystal” refers to a composition comprising a co-complex, as defined above, in crystalline form. Co-crystals include native co-crystals and heavy-atom derivative co-crystals.

When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements.

As used herein, the following definitions shall apply unless otherwise indicated. For purposes of this invention, the chemical elements are identified in accordance with the Periodic Table of the Elements, CAS version, and the Handbook of Chemistry and Physics, 75th Ed. 1994. Additionally, general principles of organic chemistry are described in “Organic Chemistry,” Thomas Sorrell, University Science Books, Sausalito: 1999, and “March's Advanced Organic Chemistry,” 5th Ed., Smith, M. B. and March, J., eds. John Wiley & Sons, New York: 2001, the entire contents of which are hereby incorporated by reference.

As used herein, the following definitions shall apply unless otherwise indicated. For purposes of this invention, the chemical elements are identified in accordance with the Periodic Table of the Elements, CAS version, and the Handbook of Chemistry and Physics, 75th Ed. 1994. Additionally, general principles of organic chemistry are described in “Organic Chemistry,” Thomas Sorrell, University Science Books, Sausalito: 1999, and “March's Advanced Organic Chemistry,” 5th Ed., Smith, M. B. and March, J., eds. John Wiley & Sons, New York: 2001, the entire contents of which are hereby incorporated by reference.

The term “alkyl” as used herein alone or as part of a group refers to saturated monovalent hydrocarbon radicals having straight or branched hydrocarbon chains or, in the event that at least 3 carbon atoms are present, cyclic hydrocarbons or combinations thereof and contains 1 to 20 carbon atoms (C₁₋₂₀alkyl), suitably 1 to 10 carbon atoms (C₁₋₁₀alkyl), preferably 1 to 8 carbon atoms (C₁₋₈alkyl), more preferably 1 to 6 carbon atoms (C₁₋₄alkyl), and even more preferably 1 to 4 carbon atoms (C₁₋₄alkyl). Examples of alkyl radicals include methyl, ethyl, propyl, isopropyl, n-butyl, isobutyl, sec-butyl, tert-butyl, pentyl, isoamyl, hexyl, cyclopropyl, cyclobutyl, cyclopentyl, cyclohexyl, and the like.

The term “alkenyl” as used herein alone or as part of a group refers to monovalent hydrocarbon radicals having a straight or branched hydrocarbon chains having one or more double bonds and containing from 2 to about 18 carbon atoms, preferably from 2 to about 8 carbon atoms, more preferably from 2 to about 5 carbon atoms. Examples of suitable alkenyl radicals include ethenyl, propenyl, alkyl, 1,4-butadienyl, and the like.

The term “alkynyl” as used herein alone or as part of a group refers to monovalent hydrocarbon radicals having a straight or branched hydrocarbon chains having one or more triple bonds and containing from 2 to about 10 carbon atoms, more preferably from 2 to about 5 carbon atoms. Examples of alkynyl radicals include ethynyl, propynyl, (propargyl), butyny,l and the like.

The term “aryl” as used herein, alone or as part of a group, includes an organic radical derived from an aromatic hydrocarbon by removal of one hydrogen, and includes monocyclic and polycyclic radicals, such as phenyl, biphenyl, naphthyl.

The term “alkoxy” as used herein, alone or as part of a group, refers to an alkyl ether radical wherein the term alkyl is as defined above. Examples of alkyl ether radical include methoxy, ethoxy, n-propoxy, isopropoxy, n-butoxy, isobutoxy, sec-butoxy, tert-butoxy, and the like.

The term “cycloalkyl” as used herein, alone or in combination, means a saturated or partially saturated monocyclic, bicyclic or tricyclic alkyl radical wherein each cyclic moiety contains from about 3 to about 8 carbon atoms, more preferably from about 3 to about 6 carbon atoms. Examples of such cycloalkyl radicals include cyclopropyl, cyclobutyl, cyclopentyl, cyclohexyl, and the like.

The term “cycloalkylalkyl” as used herein, alone or in combination, means an alkyl radical as defined above which is substituted by a cycloalkyl radical as defined above. Examples of such cycloalkylalkyl radicals include cyclopropylmethyl, cyclobutyl-methyl, cyclopentylmethyl, cyclohexylmethyl, 1-cyclopentylethyl, 1-cyclohexylethyl, 2-cyclopentylethyl, 2-cyclohexylethyl, cyclobutylpropyl, cyclopentylpropyl, cyclohexylbutyl, and the like.

The term “substituted” as used herein means that one or more of the hydrogen atoms bonded to carbon atoms in the chain or ring have been replaced with other substituents. Suitable substituents include monovalent hydrocarbon groups including alkyl groups such as methyl groups and monovalent heterogeneous groups including alkoxy groups such as methoxy groups.

The term “unsubstituted” as used herein means that the carbon chain or ring contains no other substituents other than carbon and hydrogen.

The term “branched” as used herein means that the carbon chain is not simply a linear chain. “Unbranched” means that the carbon chain is a linear carbon chain.

The term “saturated” as used herein means that the carbon chain or ring does not contain any double or triple bonds. “Unsaturated” means that the carbon chain or ring contains at least one double bond. An unsaturated carbon chain or ring may include more than one double bond.

The term “hydrocarbon group” means a chain of 1 to 25 carbon atoms, suitably 1 to 12 carbon atoms, more suitably 1 to 10 carbon atoms, and most suitably 1 to 8 carbon atoms. Hydrocarbon groups may have a linear or branched chain structure. Suitably the hydrocarbon groups have one branch.

The term “carbocyclic group” means a saturated or unsaturated hydrocarbon ring. Carbocyclic groups are not aromatic. Carbocyclic groups are monocyclic or polycyclic. Polycyclic carbocyclic groups can be fused, spiro, or bridged ring systems. Monocyclic carbocyclic groups contain 4 to 10 carbon atoms, suitably 4 to 7 carbon atoms, and more suitably 5 to 6 carbon atoms in the ring. Bicyclic carbocyclic groups contain 8 to 12 carbon atoms, preferably 9 to 10 carbon atoms in the rings.

The term “heteroatom” means an atom other than carbon e.g., in the ring of a heterocyclic group or the chain of a heterogeneous group. Preferably, heteroatoms are selected from the group consisting of sulfur, phosphorous, nitrogen and oxygen atoms. Groups containing more than one heteroatom may contain different heteroatoms.

The term “heterocyclic group” means a saturated or unsaturated ring structure containing carbon atoms and 1 or more heteroatoms in the ring. Heterocyclic groups are not aromatic. Heterocyclic groups are monocyclic or polycyclic. Polycyclic heteroaromatic groups can be fused, spiro, or bridged ring systems. Monocyclic heterocyclic groups contain 4 to 10 member atoms (i.e., including both carbon atoms and at least 1 heteroatom), suitably 4 to 7, and more suitably 5 to 6 in the ring. Bicyclic heterocyclic groups contain 8 to 18 member atoms, suitably 9 or 10 in the rings.

The term “imine” or “imino”, as used herein, unless otherwise indicated, includes a functional group or chemical compound containing a carbon-nitrogen double bond. The expression “imino compound”, as used herein, unless otherwise indicated, refers to a compound that includes an “imine” or an “imino” group as defined herein.

The term “hydroxyl”, as used herein, unless otherwise indicated, includes —OH.

The terms “halogen” and “halo”, as used herein, unless otherwise indicated, include a chlorine, chloro, Cl; fluorine, fluoro, F; bromine, bromo, Br; or iodine, iodo, or I.

The term “cyano”, as used herein, unless otherwise indicated, includes a —CN group.

The term “alcohol”, as used herein, unless otherwise indicated, includes a compound in which the hydroxyl functional group (—OH) is bound to a carbon atom. In particular, this carbon center should be saturated, having single bonds to three other atoms.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Structural Basis for Usher Activation and Intramolecular Subunit Transfer in P Pilus Biogenesis in E. coli

The goal of this study was to elucidate the intricate workings of the molecular machine that catalyzes CUP pilus assembly and opens the door for the development of potent inhibitors to block pilus biogenesis. Chaperone-usher pathway (CUP) pili are extracellular proteinaceous fibers ubiquitously found on Gram-negative bacteria, and mediate host-pathogen interactions and biofilm formation critical in pathogenesis in numerous human diseases (Nuccio S P et al., Microbiol Mol Biol Rev 71, 551-575, (2007)). During pilus assembly an outer membrane (OM) macromolecular machine called the usher catalyzes pilus biogenesis from the individual subunits that are delivered as chaperone-subunit complexes in the periplasm. The usher orchestrates pilus assembly using all five functional domains: a 24-stranded transmembrane β-barrel translocation domain (TD), a β-sandwich plug domain (PD), an amino-terminal periplasmic domain (NTD) and two carboxy-terminal periplasmic domains (CTD1 and CTD2) (Ford B et al., J Bacteriol 192, 1824-1831, (2010); Nishiyama Metal., EMBO J 24, 2075-2086, (2005); Phan G et al., Nature 474, 49-53, (2011); Remaut H et al., Cell 133, 640-652, (2008); and Thanassi D G et al., J Bacteriol 184, 6260-6269 (2002)).

Gram-negative bacteria assemble CUP pili to mediate adhesion to host and environmental surfaces, facilitate invasion into host tissues, and promote formation of intra- and extra-cellular biofilm communities. This has been best studied for urinary tract infections (UTIs), which affect 60% of women in their lifetime (Foxman B, Am J Med 113 Suppl 1A, 5S-13S (2002); and Foxman B, Dis Mon 49, 53-70, (2003)). Escherichia coli (E. coli) carries at least 38 CUP pili in its pan genome (Wurpel D J, et al., PLoS One 8, e52835, (2013)). Uropathogenic E. coli (UPEC), the causative agent of 85% of community-acquired UTIs, utilizes type 1 and P pili to mediate host- and tissue-specific adherence critical in cystitis and pyelonephritis, respectively (Ronald A, Dis Mon 49, 71-82, (2003); Ronald A R et al., Int J Antimicrob Agents 17, 343-348 (2001); Melican K et al., PLoS Pathog 7, e1001298, (2011)).

In CUP pilus assembly, individual pilus subunits or pilins are first exported across the inner membrane to the periplasm where they are guided to the OM usher via the chaperone (Lindberg F et al., J Bacteriol 171, 6052-6058 (1989); and Hultgren S J et al. Proc Natl Acad Sci USA 86, 4357-4361 (1989)). Each pilin adopts an incomplete immunoglobulin (Ig)-like structure lacking a seventh C-terminal β-strand. In a process termed donor-strand complementation (DSC), the chaperone, a boomerang shaped protein comprised of two complete mg-like domains, provides in trans its G1 β-strand to complete the pilin's mg-like fold in a non-canonical fashion (Choudhury D et al., Science 285, 1061-1066 (1999); and Sauer F G et al. Science 285, 1058-1061 (1999)). Chaperone-pilin complexes are then guided to the OM usher, a β-barrel channel that catalyzes subunit-subunit interactions through a reaction called donor-strand exchange (DSE) wherein an N terminal extension on every subunit completes the canonical Ig fold of its neighboring subunit in a zip-in zip-out mechanism that drives the dissociation of the chaperone (Zavialov A V et al. Cell 113, 587-596 (2003); Remaut H et al. Molecular cell 22, 831-842, (2006); and Sauer F G et al., Cell 111, 543-551 (2002)).

DSE events are coordinated by the PapC usher and require all five functional domains of the usher for productive interactions between pilus subunits during fiber polymerization (Geibel S et al., Nature 496, 243-246, (2013)). In biolayer interferometry (BLI) studies, the isolated NTD binds the chaperone-adhesin PapDG complex with the highest affinity relative to other chaperone-subunit complexes. Thus, P pilus assembly is thought to begin with the recruitment of PapDG to the NTD of apo-PapC. Unlike the NTD, the PD and the PD-NTD complex bind the chaperone and all chaperone-subunit complexes with nearly equal affinity (Volkan E et al. Proc Natl Acad Sci USA 109, 9563-9568, (2012)). Based on studies in the type 1 pilus system, chaperone-subunit complexes are transferred from the NTD to the CTD by an unknown mechanism (Geibel S et al., Nature 496, 243-246, (2013)). The PapC CTD2 has been shown to promote the dissociation of PapDG from the NTD in vitro. In BLI studies, CTD2 binds to all tested chaperone-subunit complexes except the PapDH complex. Incorporation of PapH results in termination of pilus biogenesis. Thus, the CTDs are thought to facilitate the hand-off of chaperone-subunit complexes from the NTD to the CTDs by an unknown mechanism, resulting in their association at the usher CTDs during activation of the usher for the initiation of pilus assembly and all subsequent pilus assembly elongation steps. Usher activation is defined as a complex multi-step mechanism, which begins with the interactions between the chaperone-adhesin and usher NTD and results in: i) its association with NTD in the periplasm; ii) transfer of chaperone-adhesin from the NTD to CTDs and; iii) Plug domain displacement.

The present work provides an X-ray crystal structure of the ternary PapC-PapDG complex in the process of transitioning from an inactive to a post-activation state, termed here a pre-activation state, delineating detailed molecular interactions between PapDG and the NTD and CTD2 of PapC. Addition of the PapDF chaperone-pilin complex to the pre-activated PapC-PapDG complex results in DSE between PapG and PapF. Mutations in the NTD-CTD2 interface significantly reduced pilus assembly in vivo and diminished the ability of PapC to promote PapG-PapF DSE in vitro. Together with functional characterization and comparison with the post-activation structures in the type 1 pilus system, the results provided herein reveal critical details about the molecular mechanism of usher activation and chaperone-adhesin translocation from the usher NTD to CTD.

The ushers that assemble the most well characterized CUP pili, the type 1 pilus and P pilus, have a sequence identity and similarity of 31% and 43%, respectively, but exhibit high structural similarity and are thought to share common assembly mechanisms. Thus, genetic, biochemical, and biophysical studies conducted in either pilus system are often combined to elucidate the molecular determinants of usher function during pilus biogenesis. However, variations in pilus assembly mechanisms have been noted in different pilus systems. In type 1 pilus biogenesis, the FimH adhesin is sufficient to activate the FimD usher to initiate pilus assembly (Nishiyama M. et al., Science 320, 376-379, (2008); Saulino E T et al., EMBO J 17, 2177-2185, (1998)). In marked contrast, in the P pilus system, efficient activation of the PapC usher involves a concerted mechanism involving both the PapG adhesin and the subsequent pilus subunit and adaptor protein, PapF (Li Q et al., Mol Microbiol 76, 159-172, (2010); Jacob-Dubuisson F et al., EMBO J 12, 837-847 (1993); and Lee Y M, et al., J Bacteriol 189, 5276-5283, (2007)).

### Results

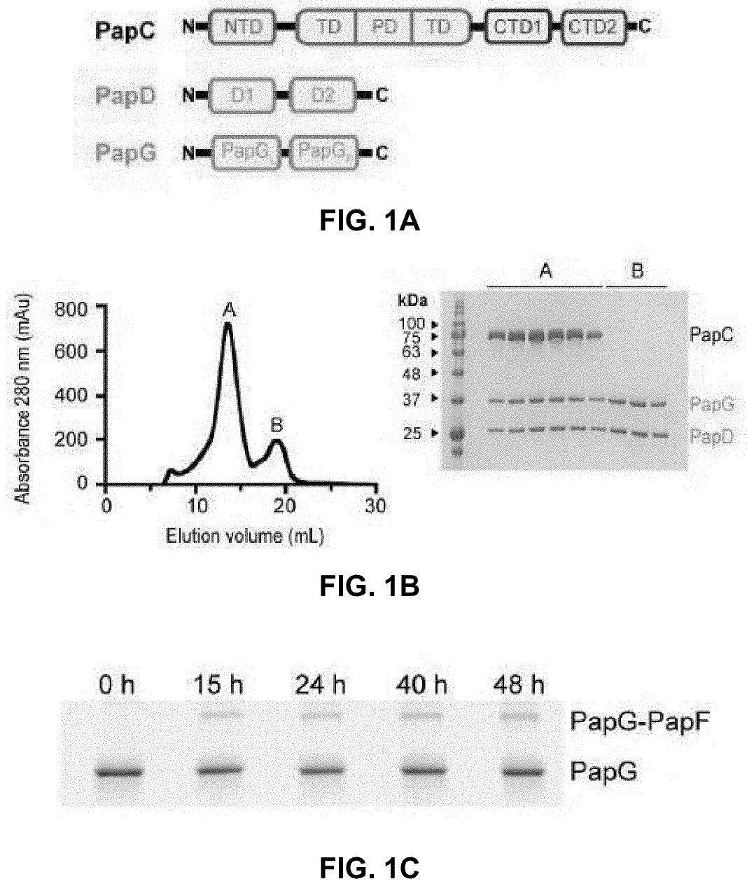

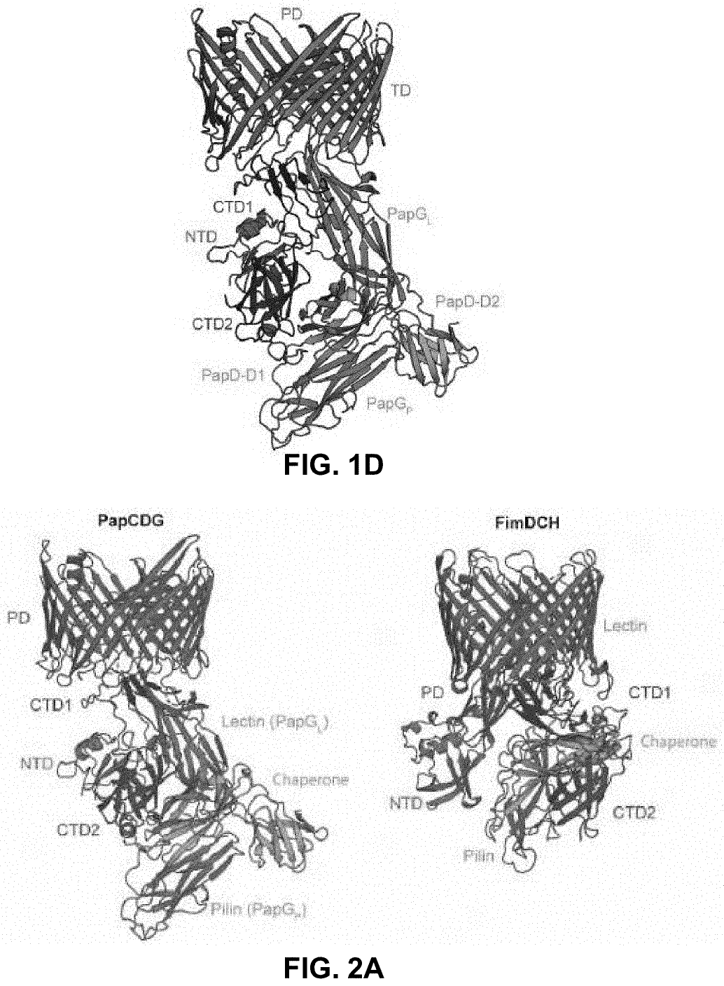

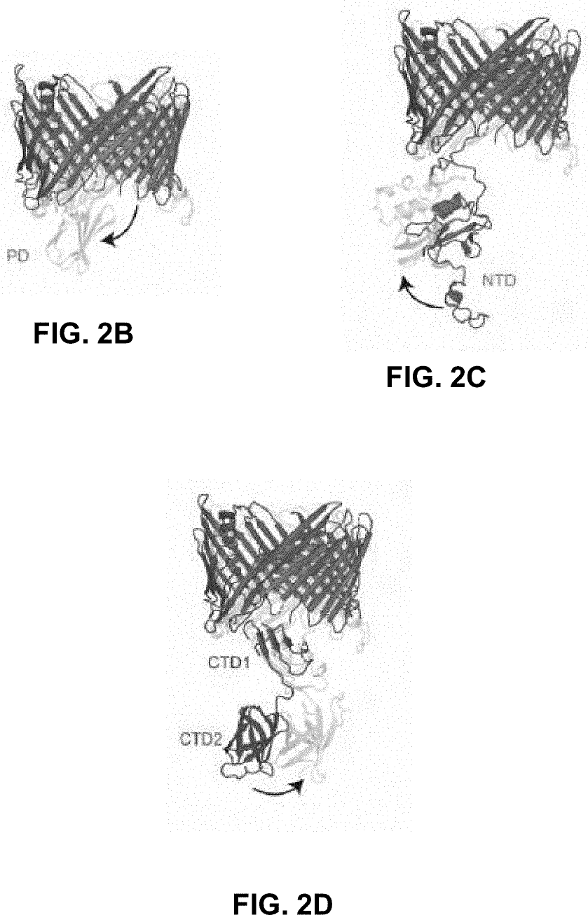

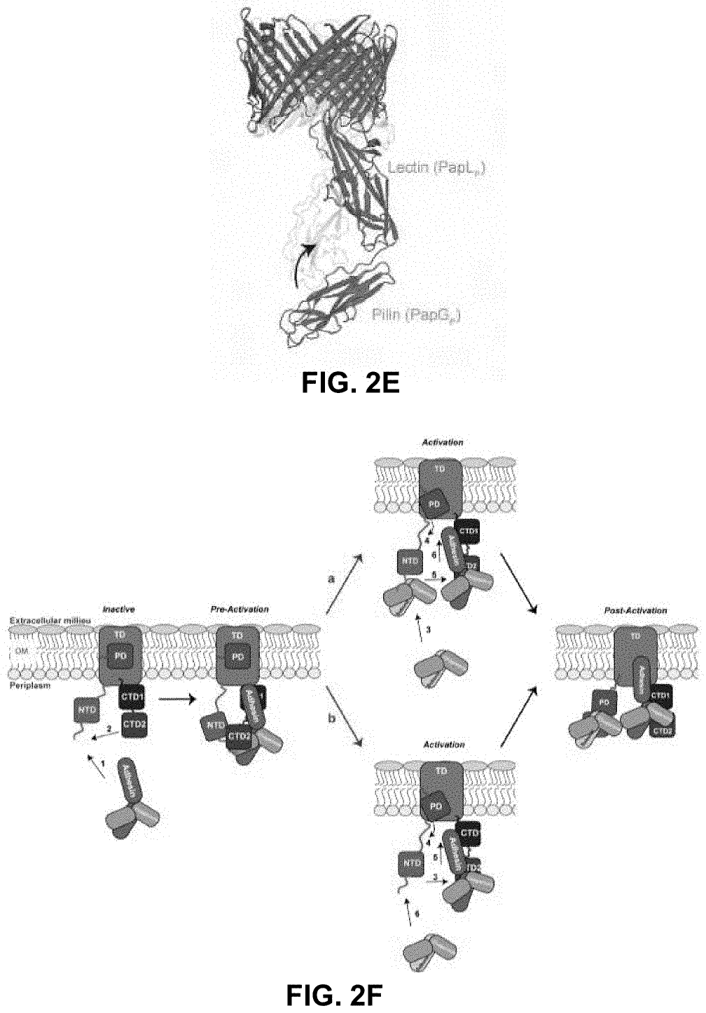

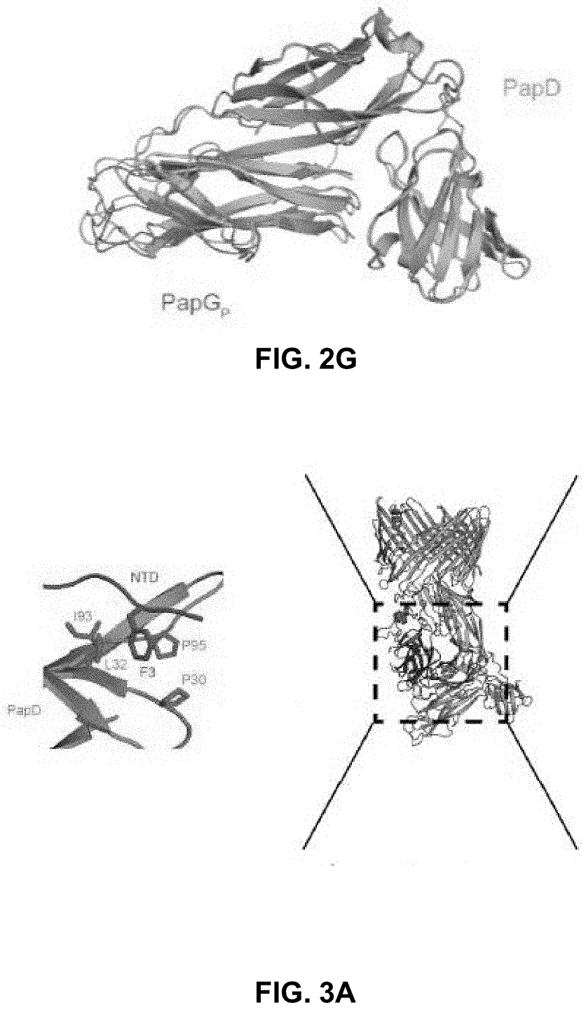

In order to elucidate the mechanism by which PapC is primed for activation and its transition to a post-activation state the following was performed: i) reconstitution of a ternary PapC-PapDG complex in vitro without PapF (FIG. 1A and FIG. 1B); ii) crystallization of the ternary PapC-PapDG complex and; iii) determination of the crystal structure at 3.7 Å resolution (FIG. 1D). To demonstrate the activity of the isolated PapC-PapDG complex a DSE assay was used to determine if the usher in the PapC-PapDG complex could promote DSE between PapG and PapF upon addition of the PapDF complex (FIG. 1C). Indeed, appearance of an SDS stable PapGF band appeared over time, albeit slowly, signifying that the purified PapC-PapDG ternary complex is capable of promoting DSE in vitro. In the time course tested, the DSE reaction between PapG and PapF does not achieve 100% completion, but this is consistent with the reported DSE rate between PapG and PapF (˜50% completion in 120 hours) (Rose R J et al., Proc Natl Acad Sci USA 105, 12873-12878 (2008); and Verger D et al. Structure 16, 1724-1731 (2008)). Moreover, the sample at the end of the time course could be purified and was demonstrated to have formed a quaternary PapC-PapDFG complex. Though it is likely that the reconstituted PapC-PapDG complex exists as a mixture of pre-activated and post-activated conformations, the crystal structure reveals a pre-activated usher state transitioning to a post-activation state in which: i) the PD still resides within the β-barrel lumen and; ii) the NTD and CTDs interact with each other and both are also engaged in binding PapDG (FIG. 1D and FIG. 2A-2B). This is in sharp contrast to the previously determined crystal structures of usher-chaperone-adhesin FimD-FimCH, and usher-tip fibrillum, FimD-FimCFGH complexes from the type 1 pilus system, in which the FimD usher has already transitioned to a post-activation state. In the FimD-FimCH and FimD-FimCFGH structures: i) the PD of FimD has translocated into a periplasmic NTD-PD complex; ii) the chaperone-subunit complex has been transferred to the CTDs and; iii) the adhesin lectin domain (FimHL) is inserted into the β-barrel pore (FIG. 2A). Notably, the FimD-FimCH structure was achieved by limited proteolysis with trypsin prior to crystallization, which resulted in cleavage of the N-terminal tail of the FimD usher, a critical motif for binding the chaperone-adhesin complex and subsequent chaperone-subunit complexes. Since critical NTD tail was removed in that study, the NTD-chaperone-adhesin and NTD-CTD2 interactions elucidated in our wild-type PapC-PapDG structure, in which the N-terminal tail of the PapC usher is critically engaged with both the chaperone-adhesin and in the NTD-CTD2 interface, could not be observed. Thus, it also raises the possibility that all (full-length) ushers, when incubated with their cognate chaperone-adhesin complex, remain in a pre-activation state until an additional subunit is recruited to the usher, competing for the N-terminal tail residues to release the chaperone-adhesin from the NTD to undergo complete translocation to the CTDs and enter the TD lumen (FIG. 2F).

In the PapC-PapDG structure, both the NTD and CTD2 are in substantially different positions as compared with the previous type 1 pilus post-activation structures (FIG. 2C-2D). These large domain rearrangements are made possible by flexible linkers connecting the NTD to the TD and CTD2 to CTD1. CTD1 remains stationary between the pre-activation and post-activation states, suggesting that CTD2 is the dynamic component of the bi-partite CTD of the usher. The PapDG complex in our structure has an essentially identical structure to isolated PapDG (FIG. 2G). The adhesin lectin domain (PapG_{L}) resides at the base of the β-barrel, burying ˜490 Å of surface area, and does not interact with any periplasmic domains of the usher (FIG. 2E). The adhesin pilin domain (PapG_{P}) moves along the same rotational axis as CTD2 (FIG. 2E). The initial binding of the PapC usher NTD and the PapDG chaperone-adhesin complex does not immediately elicit the PD displacement that primes the β-barrel for pilus extrusion through the pore. Conceptually, the PD displacement may occur concurrently with recruitment of the subsequent chaperone-subunit complex, PapDF, to the NTD and translocation of PapDG to the CTDs (FIG. 2F). Alternatively, complete translocation of PapDG to the CTDs might elicit PD displacement followed by recruitment of PapDF to an NTD-PD complex (FIG. 2F).

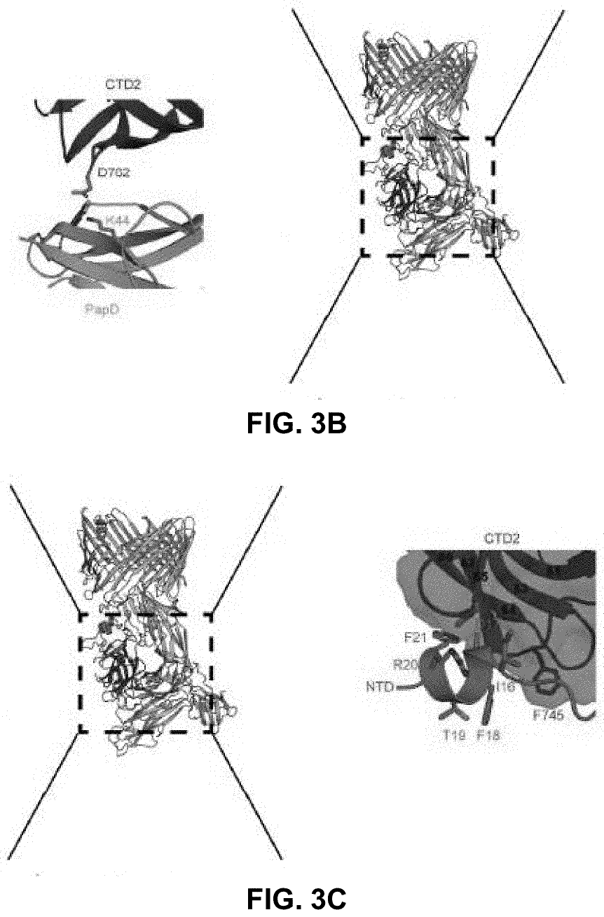

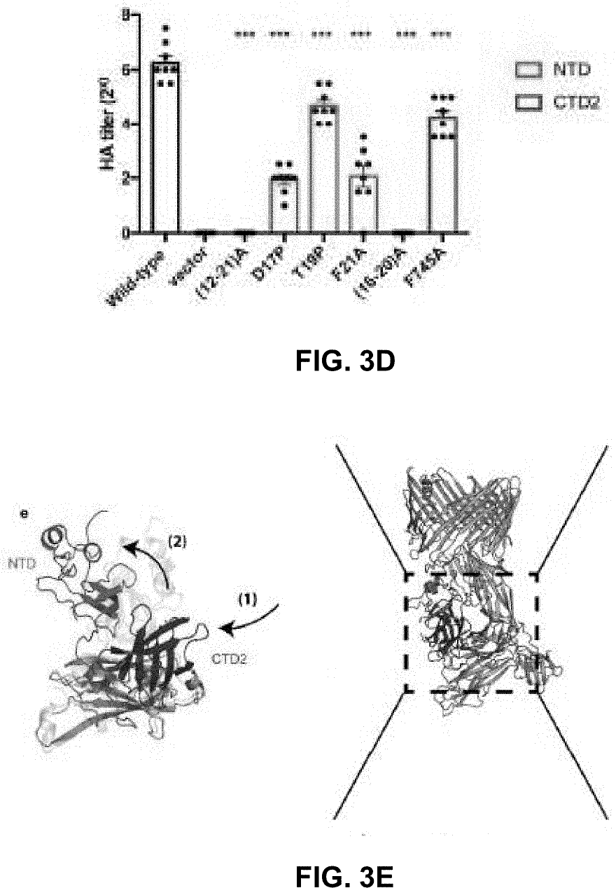

While the PD resides within the pore lumen, the periplasmic domains make extensive contacts with PapDG as well as with each other, giving rise to a pre-activated usher in the process of activation. The PapC NTD interacts, via the first eleven amino acids of its N-terminal tail, with PapD (FIG. 3A). In particular, the conserved F3 of PapC plugs into a hydrophobic pocket comprised of residues P30, L32, 193, and P95 of PapD (FIG. 3A). Mutation of F3 of the usher or L32 and 193 of the chaperone abolishes pilus assembly, suggesting that this interface is critical in usher function (Remaut H et al., Cell 133, 640-652, (2008); and Ng T W et al., J Bacteriol 186, 5321-5331, (2004)). Additionally, the usher CTD2 also contacts PapD, with D762 of CTD2 forming a salt bridge with K44 of PapD, an interaction also observed in the FimD-FimCH and FimD-FimCFGH structures (FIG. 3B).

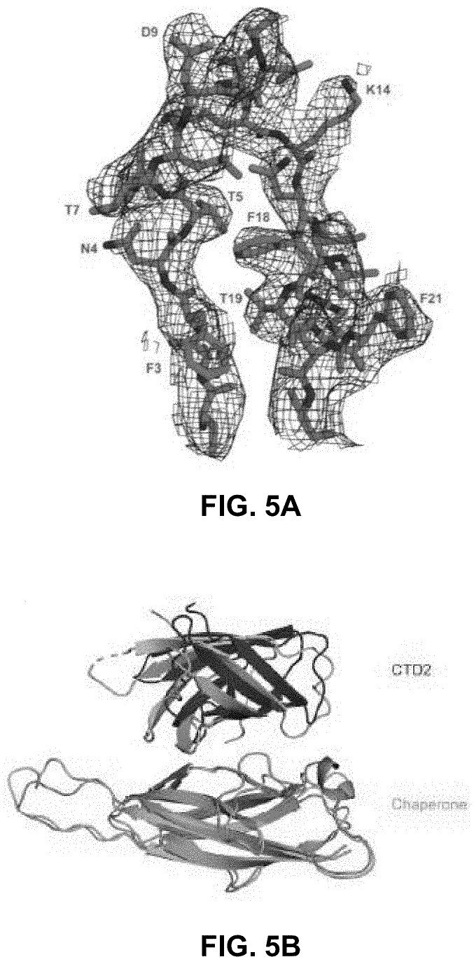

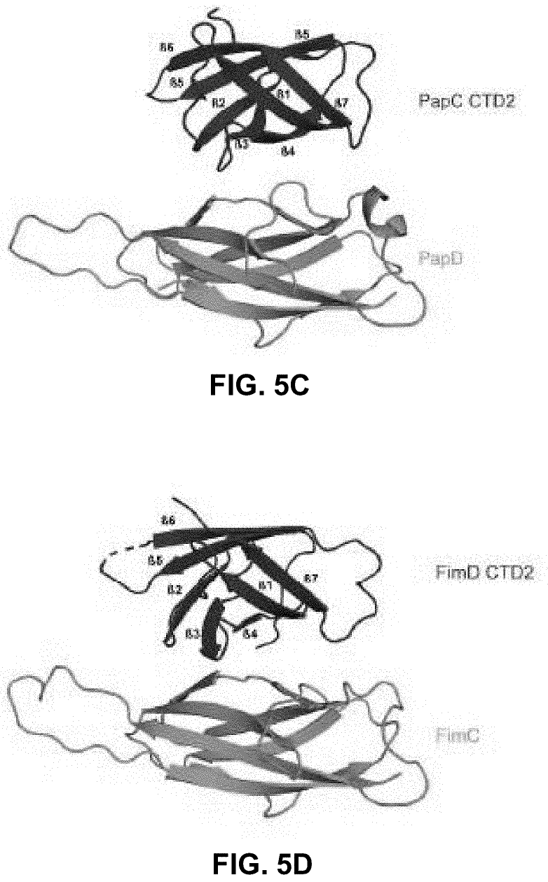

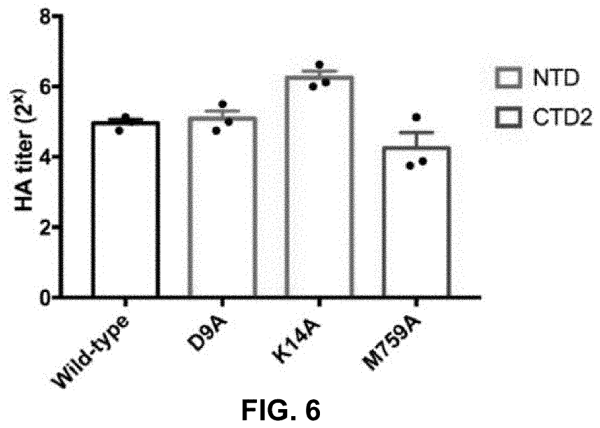

Remarkably, direct intra-usher interactions between the usher NTD and CTD2 are also observed at the NTD-PapDG-CTD2 interface (FIG. 3C). Despite the medium resolution of the structure, the N-terminal tail residues 12-21 of the NTD are well resolved in the electron density map (FIG. 5A) and form extensive interactions with the bottom cleft of CTD2, particularly the β1-β2 and β5-β6 loops in CTD2 (FIG. 3C). While the interactions between CTD2 and the chaperone are observed in the post-activation FimD-FimCH structure (FIG. 5B-5D), the NTD-CTD2 interface is an unprecedented, unique feature in our pre-activation PapC-PapDG structure. This interface was not observed in the FimD-FimCH structure because the NTD motif was proteolytically removed by trypsin to aid in crystallization. To evaluate the physiological relevance of this NTD-CTD2 interface, we made multiple mutations in amino acids comprising this interface, with a focus on an N-terminal α-helix in the usher NTD that is positioned directly next to CTD2 (FIG. 3C). To investigate their functional importance, we tested the ability of each PapC mutant to complement a ΔpapC pap operon (papAHDJKEFG) in a hemagglutination assay, which is a measure of overall levels of piliation on the bacterial surface (HA titer analysis) (FIG. 3D). All mutations in this region, especially mutants destabilizing the α-helix, resulted in a significant reduction in HA titer, ranging from a complete ablation of HA titer to a 2- to 16-fold decrease in HA titer (FIG. 3D). These defects in P pilus assembly are specific to this interface as mutations of surrounding residues on the N-terminal tail or CTD2 have no effect (FIG. 6A).

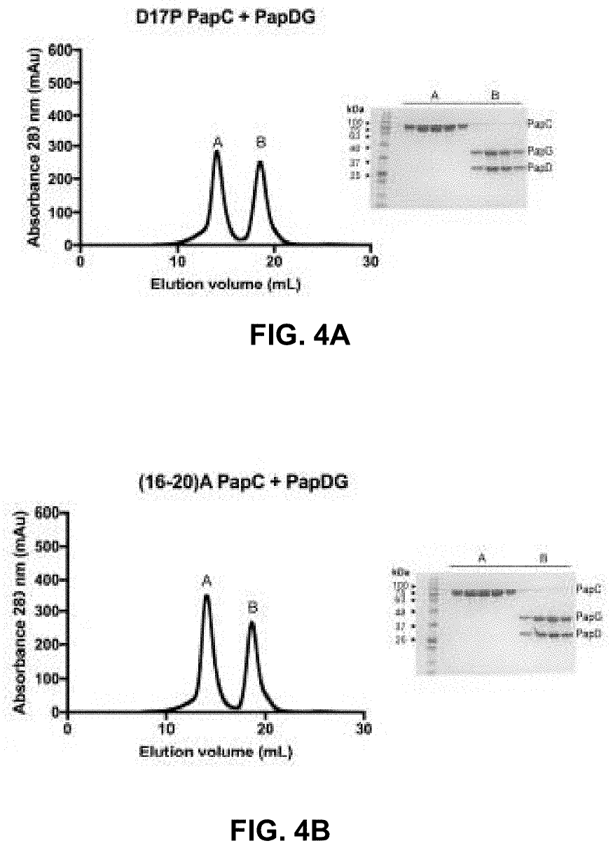

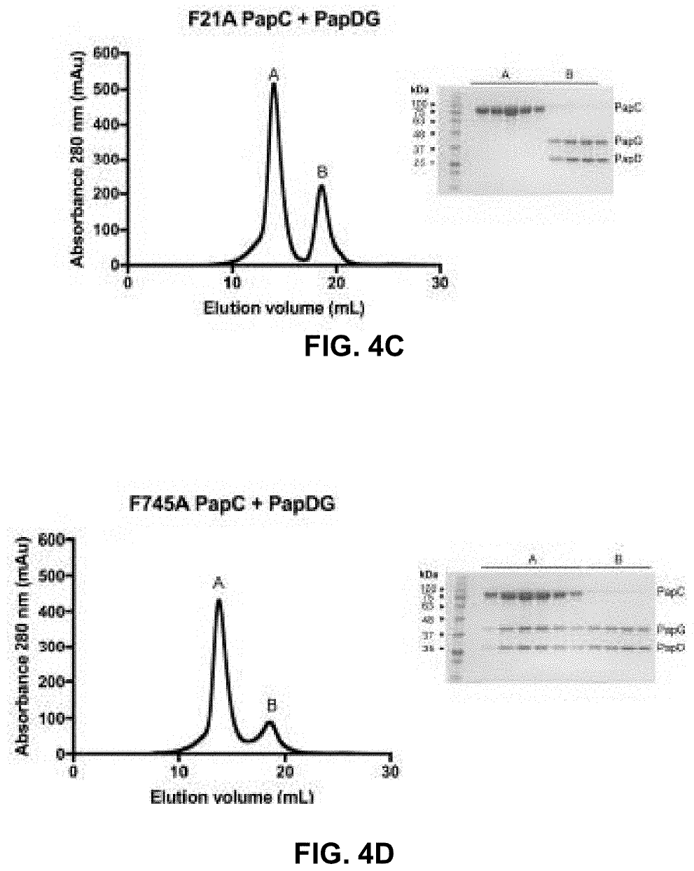

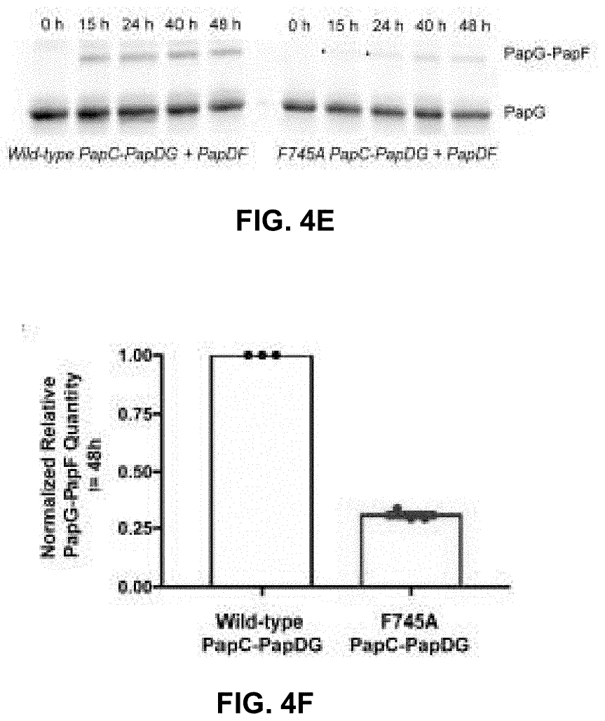

To further interrogate the role of this interface in usher activation, we selected four PapC mutants (D17P, (16-20)A, F21A, and F745A) and tested their ability to first form a stable ternary complex with PapDG and then to promote DSE between PapG and PapF. Mutations destabilizing the N-terminal α-helix (D17P or (16-20)A) or a residue flanking the end of the α-helix (F21A) resulted in an inability of the mutant PapC ushers to form a stable ternary complex in vitro (FIG. 4A-4C). Interestingly, the F21A PapC mutant had been previously characterized as exhibiting an HA titer defect and reduced association with PapDG, and was proposed to be involved in subunit discrimination during pilus assembly. In light of our structure, it appears that this residue may play a role in stabilizing an NTD-CTD2 interface during a pre-activation usher state. Contrary to the NTD mutants, the CTD2 mutant (F745A) retained its ability to form a stable ternary complex with PapDG in vitro but was not able to efficiently promote DSE between PapG and PapF relative to wild-type PapC-PapDG (FIG. 4D-4F). Taken together, these results demonstrate that the NTD-CTD2 interaction is essential for stable association of PapDG with the PapC usher and translocation of subunits from the NTD to CTDs, further suggesting that our wild-type PapC-PapDG structure represents a productive on-pathway intermediate rather than an off-pathway conformation owing to detergent solubilization and crystallization, although additional studies will be required to address this possibility. Moreover, our structure-function analysis of PapC-PapDG suggests that CTD2 drives subunit transfer, potentially by using the avidity of its interactions with both the NTD and PapD to competitively displace subunits from the NTD during pilus biogenesis (FIG. 3E). Several lines of evidence further support this competitive displacement mechanism. First, previous BLI studies have shown that in isolation CTD2 can promote dissociation of a chaperone-adhesin bound to the NTD. Second, it has been shown in the type 1 pilus system that upon usher activation the CTDs are the high affinity binding site (K_{d}=40 nM) in the full-length usher compared to the NTD (K_{d}=389 nM). Therefore, these data suggest this differential affinity potentially drives translocation from the NTD to the CTDs, and our structure further demonstrates allosteric destabilization of the chaperone-subunits at the NTD may be facilitated by a direct interaction between the NTD and CTD2.

The chaperone-usher pathway represents an ideal system in which to study the mechanisms by which binding and allosteric interactions drive sequential steps in a multi-domain assembly machine without ATP or other energy inputs. This work provides insight into the mechanism by which the outer membrane usher transfers substrates from its N-terminus to C-terminus via an unexpected interaction between two periplasmic domains. Previously, bicyclic 2-pyridone compounds termed pilicides were determined to be inhibitors of pilus biogenesis by specifically blocking chaperone-subunit interactions with the N-terminal tail of the usher NTD. In light of our structure, structure-activity relationship (SAR) analysis could be utilized to design even more potent inhibitors that bind to this critical chaperone-usher interface. Furthermore, our structure potentially provides a template for the design of novel small molecules that sterically block the NTD-CTD2 interface to selectively hinder the usher from acting as an assembly platform for the development of antibiotic-sparing therapeutics. In summary, the PapC-PapDG structure and analysis of its activity in vitro, provides an extraordinary view of a bacterial nanomachine caught in the act of being primed to catalyze macromolecular protein assembly across the outer membrane. Moreover, this structure may facilitate the development of new drugs that block chaperone-adhesin translocation to prevent assembly of one of the bacterium's major virulence factors.

### Methods

### Expression and Purification of the Full-Length Wild-Type PapC Usher.

Plasmid pDG2 encoding wild-type PapC with a thrombin cleavage site and a 6×HIS tag added to the C-terminus and the multi-porin mutant BL21(DE3)Omp8 E. coli strain were used for PapC expression as previously described (Henderson N S et al., Methods Mol Biol 966, 37-52, (2013)). For protein expression, E. coli was grown at 37° C. with aeration in LB medium supplemented with 100 μg/mL ampicillin and was induced at an OD600 of 0.6-0.8 for 2 hours at 37° C. by the addition of 0.1% L-arabinose. To isolate the outer membrane (OM) fraction, resuspended cells supplemented with 1×EDTA-free protease inhibitor cocktail (Pierce) and DNAse I were subjected to microfluidizer cell disruption via 4 passes at 30 kPsi. Unbroken cells were removed by centrifugation at 4,500×g for 10-12 minutes at 4° C. Lysed cells were centrifuged at 39,000×g for 60 minutes at 4° C. to pellet total membranes. Total membranes were resuspended using a dounce homogenizer and extracted with 1% Sarkosyl by stirring at 25° C. for 45 minutes. OM was pelleted by centrifugation at 39,000×g for 60 minutes at 4° C. and then was solubilized by resuspension using a dounce homogenizer in 20 mM Tris-HCl pH 8.0, 300 mM NaCl, 1× Pierce Protease Inhibitor Cocktail, and 1% dodecyl-maltopyranoside (DDM, Sol-grade; Anatrace) and stirring overnight at 4° C. The OM extract was clarified by centrifugation at 39,000×g for 30 minutes at 4° C. The clarified OM extract was added to 3 mL nickel affinity resin equilibrated with 20 mM Tris-HCl pH 8.0, 300 mM NaCl, 0.2% DDM, 20 mM imidazole (Buffer A). OM extract/nickel resin slurry was stirred slowly at 4° C. for 3 hours to allow batching binding of PapC to nickel affinity resin. Batch bound resin was added to a 30 mL polypropylene column and washed with 10 column volumes (CV) of Buffer A. PapC was eluted with 3 CV 20 mM Tris-HCl pH 8.0, 100 mM NaCl, 0.1% DDM, 400 mM imidazole (Buffer B).

### Expression and Purification of the PapDG Chaperone-Adhesin Complex.

Plasmids pDF1 (encoding PapD and pTrcGII (encoding PapG) and the E. coli C600 strain were used for PapDG expression (Sauer F G et al., Cell 111, 543-551 (2002); and Dodson K W et al. Cell 105, 733-743 (2001)). Periplasm was prepared as described previously (Hultgren S J et al. Proc Natl Acad Sci USA 86, 4357-4361 (1989)). Periplasm was dialyzed into 1×PBS supplemented with 250 mM NaCl (Buffer C) and loaded onto a Cobalt HTC agarose column (Goldbio). PapDG was eluted with a linear gradient of Buffer C with 0-300 mM imidazole where the complex eluted in 210 mM imidazole. Fractions containing PapDG were pooled and dialyzed into 20 mM Tris-HCl pH 8.0, 500 mM (NH₄)₂SO₄ (Buffer D) for a second Butyl 4FF column (GE Healthcare). PapDG was eluted with a linear gradient of Buffer D at 250 mM (NH₄)₂SO₄. Fractions containing PapDG were pooled for a final Cobalt HTC agarose column (Goldbio). Using a linear gradient of Buffer C with 0-300 mM imidazole, the complex was eluted in 180 mM imidazole. The fractions containing PapDG were dialyzed into 20 mM Tris-HCl pH 8.0, 100 mM NaCl and 0.1% DDM (Sol-grade; Anatrace) for in vitro ternary complex reconstitution.

### In Vitro Reconstitution and Purification of Wild-Type and Mutant PapC-PapDG.

Affinity purified PapC (ε at 280 nm=156330 M⁻¹ cm⁻¹) and column-purified PapDG (ε at 280 nm=99615 M⁻¹ cm⁻¹) were mixed in a 1:1.2 molar ratio (2.2 μM-6.3 μM: 2.6-7.5 μM) in Buffer B and were allowed to form stable complex by rotating gently at 4° C. for 1 hour, prior to size-exclusion chromatography (SEC). The mixture was concentrated using a 100 kDa MWCO centrifugal device (Amicon), loaded onto a Superose 6 10/300 GL column (GE Healthcare), and purified at 4° C. in buffer containing 20 mM Tris-HCl pH 8.0, 50 mM NaCl, and 5 mM 5-cyclohexyl-1-pentyl-β-d-maltoside (CYMAL-5, Anagrade; Anatrace) (Buffer E). The fractions corresponding to PapC-PapDG complex were collected and concentrated using a 100 kDa MWCO centrifugal device (Amicon) for crystallization or DSE experiments.

### Donor Strand Exchange (DSE) Assay of Wild-Type and Mutant PapC-PapDG.

Crystals of the PapC-PapDG ternary complex were grown using sitting-drop vapor diffusion by mixing 0.3 μL protein with 0.3 μL reservoir solution and incubating at 20° C. The crystallization drops contained 9-11 mg/mL (58.4-71.4 μM) of purified complex (c at 280 nm=255945 M⁻¹ cm⁻¹), 50 mM sodium citrate pH 5.0-6.0, 50 mM lithium sulfate, 50 mM sodium sulfate and 7-14% PEG 4000. Crystals of two distinct morphologies appeared in the same drops, only plate-like crystals diffracted X-rays beyond 4 Å resolution. Crystals appeared in one week and grew to full size within two weeks. Plate-like crystals were flash-cooled in liquid nitrogen using the mother liquor supplemented with 5 mM CYMAL-5 and 30% (v/v) glycerol as cryoprotectant.

Diffraction data were collected at the Advanced Photon Source beamline 24-ID-E and were integrated and scaled using the HKL2000 package (Otwinowski Z et al., Methods Enzymol 276, 307-326 (1997)). Because of strong anisotropy the dataset was corrected using the UCLA-DOE Diffraction Anisotropy server (Strong Metal., Proc Natl Acad Sci USA 103, 8060-8065, (2006)). After truncation the resolution limit along the reciprocal cell directions a*, b*, and c* was 3.7, 4.6 and 3.7 Å, respectively. An isotropic B-factor of −47.12 Å was applied to restore the magnitude of high-resolution reflections. The crystal belonged to space group C222₁ with one PapC-PapDG complex per asymmetric unit.

Phasing was obtained by molecular replacement using Phaser with structures of the PapC translocation domain (PDB ID: 2VQI), PapD-PapG pilin domain (PDB ID: 2WMP), PapG lectin domain (PDB ID: 1J8S) and PapC CTD2 domain (PDB ID: 3L48) as search models (McCoy A J et al. J Appl Crystallogr 40, 658-674, (2007)). Cycles of model building and refinement were carried out in Coot and REFMAC before building the remaining of the complex (Emsley P et al., Acta Crystallogr D Biol Crystallogr 60, 2126-2132, (2004); and Murshudov G N et al., Acta Crystallogr D Biol Crystallogr 53, 240-255, (1997)). For model building of the PapC NTD and CTD1, the PapC NTD and CTD1 models were first generated based on the full-length FimD structure (PDB ID: 3RFZ) using the SWISS-MODEL server (Arnold K et al., Bioinformatics 22, 195-201, (2006)). The NTD model was fitted into the electron density using rigid body fit in Coot and was then manually adjusted. The PapC NTD tail (residues 1-30) and the linker between the PapC NTD and the translocation domain were manually built. At the CTD1 location there was clear electron density corresponding to three β-strands, one of which connects to CTD2. Based on this observation, the CTD1 model was placed into the electron density and was then manually adjusted. The assignment of the sequence register for the PapC NTD and CTD1 was based on the model generated from SWISS-MODEL. The complete model was iteratively adjusted and refined. At the final refinement step the TLS and “jelly body” refinement were applied when running REFMAC. The final model, refined to R_{work}/R_{free}=0.282/0.329, contains: PapC residues 1-54, 67-248, 252-347, 355-430, 435-588, 592-633, 651-662, 672-687, 714-808; PapD residues 1-215; PapG residues 1-316. Residues without sidechain density were modeled as alanine. Percentage of residues in favored, allowed and disallowed regions of the Ramachandran plot were 92%, 8%, and 0%, respectively (Volkan E et al. Proc Natl Acad Sci USA 110, 20741-20746, (2013)).

### Calculation of Domain Movements Using DynDom

Domain movements between the pre-activation and post-activation usher states were calculated using the DynDom Protein Domain Motion Analysis web server⁴⁶. DynDom requires the use of two different conformations of the same protein or protein complex. In order to create a post-activation PapC-PapDG usher model, the individual TD, NTD, and CTD2 domains were superimposed to the corresponding domains in the FimD-FimCH structure (PDB ID: 3RFZ) and each resulting superimposed domain was exported as a molecule. Merging the coordinates of TD and NTD or TD and CTD2 created models of the post-activation TD-NTD and TD-CTD2 structures, where the TD was used as the static domain and the NTD or CTD2 were considered dynamic domains. To create an equivalent model in the pre-activation usher state, TD-NTD or TD-CTD2 were exported as molecules from our solved structure. Pre-activation TD-NTD or TD-CTD2 and post-activation TD-NTD or TD-CTD2 molecules were input as conformer 1 and conformer 2, respectively. The resulting output reported the translation and rotation for the dynamic domain in question, which are described in the figure legends.

### Functional Analysis of PapC Mutants

All PapC mutants were derived from plasmid pDG2 using site-directed mutagenesis (Henderson N S et al., Methods Mol Biol 966, 37-52, (2013)). We created the following mutations: i) substitution of residues 12-21 to alanine (12-21A); ii) substitution of residues 16-20 to alanine (16-20A); iii) introduction of a proline residue at position 17 (D17P) and position 19 (T19P) to specifically disrupt the beginning and middle of the NTD N-terminal α-helix; and iv) substitution of alanine for a conserved phenylalanine (F745A) in CTD2. All constructs generated with site-directed mutagenesis methods were sequenced to verify that the correct mutations were made. The HA titer of C600 cells harboring pEV33, a Tetracycline (Tet)-resistant, tryptic soy agar (TSA)-inducible plasmid containing the entire pap operon with PapC inactivated by a XhoI linker, and each papC mutant plasmid was determined by overnight growth on selective TSA media supplemented with 100 μg/mL ampicillin, 15 μg/mL tetracycline, and 0.005% L-arabinose. HA titer assays were performed as previously described (Slonim L N et al., EMBO J 11, 4747-4756 (1992)). The HA endpoint was defined as the last dilution well before erythrocyte buttons formed. Titers are represented as the reciprocal of the endpoint dilution. The statistical significance of differences between wild-type and mutant PapC in experiments was determined by an unpaired two-tailed Mann-Whitney test. Statistical analyses were performed using Graphpad Prism 7.

### Data Availability

The data that support the findings of this study are available from the corresponding authors upon request. Atomic coordinates and structure factors for the reported crystal structure have been deposited into the Protein Data Bank (PDB) under accession code 6CD2 which is incorporated herein by reference in its entirety.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method of treating or preventing a bacterial infection in a subject in need thereof, the method comprising administering to the subject an effective amount of a composition comprising a compound that prevents or inhibits the interaction between an usher protein's amino-terminal periplasmic domain (NTD) and carboxy-terminal periplasmic domain 2 (CTD2).

2. The method of claim 1, wherein the compound is a bicyclic 2-pyridone compound comprising formula (I):

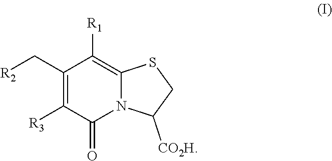

3. The method of claim 2, wherein R₁, R₂, and/or R₃ substituents directly disrupt the tripartite interface comprised of the PapC NTD, PapC CTD2 and PapD.

4. The method of claim 3, wherein the substituent at R₂ and/or R₃ directly disrupt the NTD-CTD2 interface and mimic sidechains of residues S22 and F21 from the NTD.

5. The method of claim 3, wherein the substituent at R₂ introduces steric hindrance at the NTD-CTD2 interface.

6. The method of claim 3, wherein the substituent at R₁ direct disrupts the N-terminal tail a-helix.

7. The method of claim 1, wherein the compound is a bicyclic 2-pyridone compound comprising formula (Ia): 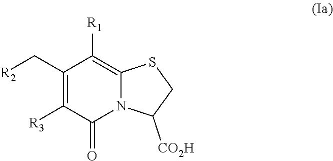
wherein:
R₁ is independently selected from H, C₃₋₆ cycloalkyl, or substituted phenyl group;
R₂ is independently selected from H or aryl; and
R₃ is independently selected from H or —CH₂—X, wherein X is a heterocyclic group.

8. The method of claim 7, wherein R₁ is

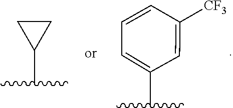

9. The method of claim 7, wherein R₂ is

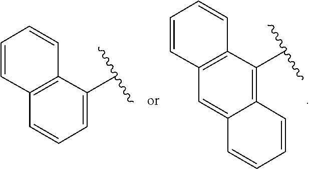 [SMILES: *c1cccc2ccccc12 ; *c1c2ccccc2cc2ccccc12]

10. The method of claim 7, wherein R₃ is H or

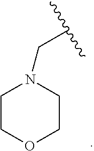 [SMILES: *CN1CCOCC1]

11. The method of claim 1, wherein the compound is

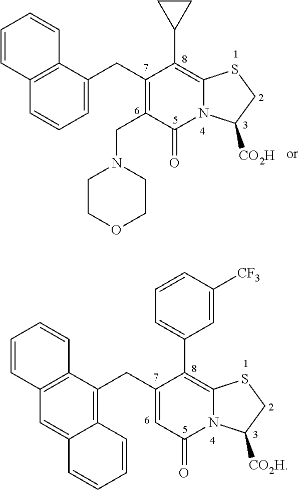 [SMILES: O=C(O)[C@@H]1CSc2c(C3CC3)c(Cc3cccc4ccccc34)c(CN3CCOCC3)c(=O)n21 ; O=C(O)[C@@H]1CSc2c(-c3cccc(C(F)(F)F)c3)c(Cc3c4ccccc4cc4ccccc34)cc(=O)n21]

12. The method of claim 1, wherein the compound blocks chaperone-adhesion translocation, thereby inhibiting pilus biogenesis.

13. The method of claim 1, wherein the method of treating an infection does not include administration of an antibiotic.

14. The method of claim 1, wherein the method of treating includes administration of an antibiotic.

15. The method of claim 1, wherein the bacteria comprises a chaperone-usher pathway pilus.

16. The method of claim 1, wherein the bacteria is a gram-negative bacteria.

17. The method of claim 1, wherein the compound prevents or inhibits the interface between residues 12-22 of NTD and CTD2.

18. The method of claim 1, wherein the compound prevents or inhibits the interface between residues 12-21 of NTD and CTD2.

19. The method of claim 1, wherein the compound mimics the side chains of residues S22 and F21 of NTD.
